SEQUENCE LISTING (54 sequences)

SEQ ID NO: 1 - DNA, 1234 bp, Homo sapiens
attccacgttcttaactgttccattttccgtatctgcttcgggcttccacctcatttttttcgctttgcccattctgtttcagccagtcgccaagaatcatgaaagtcgccagtggcagcaccgccaccgccgccgcgggccccagctgcgcgctgaaggccggcaagacagcgagcggtgcgggcgaggtggtgcgctgtctgtctgagcagagcgtggccatctcgcgctgcgccgggggcgccggggcgcgcctgcctgccctgctggacgagcagcaggtaaacgtgctgctctacgacatgaacggctgttactcacgcctcaaggagctggtgcccaccctgccccagaaccgcaaggtgagcaaggtggagattctccagcacgtcatcgactacatcagggaccttcagttggagctgaactcggaatccgaagttggaacccccgggggccgagggctgccggtccgggctccgctcagcaccctcaacggcgagatcagcgccctgacggccgaggtgagatccagatccgaccactagatcatccttataccgacggggaaacggaggccagagagggcgtgggcgcttgcaccacttccgtcccatccttgcgggtacctggctatgcgggggtgcctaaggagcctggaaaaagcgctcccccgtcgtgcttcctggggaagggggcgttcgctgcgctcggagcggcgtcccttccaacccgccggtctcatttcttctcgttttcacaggcggcatgcgttcctgcggacgatcgcatcttgtgtcgctgaagcgcctcccccagggaccggcggaccccagccatccagggggcaagaggaattacgtgctctgtgggtctcccccaacgcgcctcgccggatctgagggagaacaagaccgatcggcggccactgcgcccttaactgcatccagcctggggctgaggctgaggcactggcgaggagagggcgctcctctctgcacacctactagtcaccagagactttagggggtgggattccactcgtgtgtttctattttttgaaaagcagacattttaaaaaatggtcacgtttggtgcttctcagatttctgaggaaattgctttgtattgtatattacaatgatcaccgactgaaaatattgttttacaatagttctgtggggctgtttttttgttattaaacaaataatttagatggtgaaaaaaaaaaaa

SEQ ID NO: 2 - protein, 149 aa, Homo sapiens
MKVASGSTATAAAGPSCALKAGKTASGAGEVVRCLSEQSVAISRCAGGAGARLPALLDEQQVNVLLYDMNGCYSRLKELVPTLPQNRKVSKVEILQHVIDYIRDLQLELNSESEVGTPGGRGLPVRAPLSTLNGEISALTAEVRSRSDH

SEQ ID NO: 3 - DNA, 993 bp, Homo sapiens
attccacgttcttaactgttccattttccgtatctgcttcgggcttccacctcatttttttcgctttgcccattctgtttcagccagtcgccaagaatcatgaaagtcgccagtggcagcaccgccaccgccgccgcgggccccagctgcgcgctgaaggccggcaagacagcgagcggtgcgggcgaggtggtgcgctgtctgtctgagcagagcgtggccatctcgcgctgcgccgggggcgccggggcgcgcctgcctgccctgctggacgagcagcaggtaaacgtgctgctctacgacatgaacggctgttactcacgcctcaaggagctggtgcccaccctgccccagaaccgcaaggtgagcaaggtggagattctccagcacgtcatcgactacatcagggaccttcagttggagctgaactcggaatccgaagttggaacccccgggggccgagggctgccggtccgggctccgctcagcaccctcaacggcgagatcagcgccctgacggccgaggcggcatgcgttcctgcggacgatcgcatcttgtgtcgctgaagcgcctcccccagggaccggcggaccccagccatccagggggcaagaggaattacgtgctctgtgggtctcccccaacgcgcctcgccggatctgagggagaacaagaccgatcggcggccactgcgcccttaactgcatccagcctggggctgaggctgaggcactggcgaggagagggcgctcctctctgcacacctactagtcaccagagactttagggggtgggattccactcgtgtgtttctattttttgaaaagcagacattttaaaaaatggtcacgtttggtgcttctcagatttctgaggaaattgctttgtattgtatattacaatgatcaccgactgaaaatattgttttacaatagttctgtggggctgtttttttgttattaaacaaataatttagatggtgaaaaaaaaaa

SEQ ID NO: 4 - protein, 155 aa, Homo sapiens
MKVASGSTATAAAGPSCALKAGKTASGAGEVVRCLSEQSVAISRCAGGAGARLPALLDEQQVNVLLYDMNGCYSRLKELVPTLPQNRKVSKVEILQHVIDYIRDLQLELNSESEVGTPGGRGLPVRAPLSTLNGEISALTAEAACVPADDRILCR

SEQ ID NO: 5 - protein, 16 aa, ARTIFICIAL
  Id1 antagonistic peptide
ELLILQRLVQVILALQ

SEQ ID NO: 6 - protein, 16 aa, artificial
  peptide antagonist of Id1.
ELLFLQRLVQLILALQ

SEQ ID NO: 7 - protein, 16 aa, artificial
  peptide antagonist of Id1.
ELLFLQALVQLILALQ

SEQ ID NO: 8 - protein, 16 aa, artificial
  peptide antagonist of Id1.
ELLFLQRLVQLIEALQ

SEQ ID NO: 9 - protein, 16 aa, artificial
  peptide antagonist of Id1.
DLLLLQRLVQLIEALQ

SEQ ID NO: 10 - protein, 16 aa, artificial
  peptide antagonist of Id1.
DLLFLQRLVQLIEALQ

SEQ ID NO: 11 - protein, 16 aa, artificial
  Id1 antagonistic peptide
ELKFLQRLVQLIEALQ

SEQ ID NO: 12 - protein, 16 aa, artificial
  peptide antagonist of Id1.
ELEFLQRLVQLIEALQ

SEQ ID NO: 13 - protein, 16 aa, artificial
  peptide antagonist of Id1.
ELEFLQRLVDLIEKLQ

SEQ ID NO: 14 - protein, 16 aa, artificial
  peptide antagonist of Id1.
DLEFLQRLVDLIEKLQ

SEQ ID NO: 15 - protein, 16 aa, artificial
  peptide antagonist of Id1.
ELKFLQRLVDLIEKLQ

SEQ ID NO: 16 - protein, 16 aa, artificial
  peptide antagonist of Id1.
ELKFLQRLVDLIEKLE

SEQ ID NO: 17 - DNA, 23 bp, artificial
  Antisense for silencing Id1 expression
gcaccagctccttgaggcgtgag

SEQ ID NO: 18 - DNA, 23 bp, artificial
  Anstisense for silencing Id1 expression
gcaccagctccttgaggcgugag

SEQ ID NO: 19 - DNA, 20 bp, artificial
  Anstisense for silencing Id1 expression
ggcggcggtggcggtgctgc

SEQ ID NO: 20 - DNA, 20 bp, artificial
  Anstisense for silencing Id1 expression
gacgtgctggagaatctcca

SEQ ID NO: 21 - DNA, 20 bp, artificial
  Antisesnse for silencing Id1 expression
tgaggtggaagcccgaagca

SEQ ID NO: 22 - protein, 20 aa, artificial
  Pancreas targeting peptide
LSGTPERSGQAVKVKLKAIP

SEQ ID NO: 23 - protein, 19 aa, artificial
  Pancreas targeting peptide
GAWEARDRIAEWGSWGIPS

SEQ ID NO: 24 - protein, 9 aa, artificial
  Pancreatic islet targeting peptide
CVSNPRWKC

SEQ ID NO: 25 - protein, 9 aa, artificial
  Pancreatic islet targeting peptide
CHVLWSTRC

SEQ ID NO: 26 - protein, 11 aa, artificial
  HIV-1 TAT protein transduction domain
YGRKKRRQRRR

SEQ ID NO: 27 - protein, 17 aa, artificial
  Antennapedia protein transduction domain
GRQIKIWFQNRRMKWKK

SEQ ID NO: 28 - protein, 12 aa, artificial
  Id1 peptide antagonist
YIEGLQALLRDQ

SEQ ID NO: 29 - DNA, 22 bp, artificial
  oligonucleotide for amplifying Cyclophilin A (Ppia)
tgtgccagggtggtgactttac

SEQ ID NO: 30 - DNA, 20 bp, artificial
  oligonucleotide for amplifying Cyclophilin A (Ppia)
tgggaaccgtttgtgtttgg

SEQ ID NO: 31 - DNA, 20 bp, artificial
  oligonucleotide for amplifying Id1
ttggtctgtcggagcaaagc

SEQ ID NO: 32 - DNA, 25 bp, artificial
  oligonucleotide for amplifying Id1
gcaggtccctgatgtagtcgattac

SEQ ID NO: 33 - DNA, 21 bp, artificial
  oligonucleotide for amplifying insulin mRNA
tcttctacacacccatgtccc

SEQ ID NO: 34 - DNA, 18 bp, artificial
  oligonucleotide for amplifying insulin mRNA
ggtgcagcactgatctac

SEQ ID NO: 35 - DNA, 21 bp, artificial
  oligonucleotide for amplifying glucagon mRNA
atgaatgaagacaaacgccac

SEQ ID NO: 36 - DNA, 21 bp, artificial
  oligonucleotide for amplifying glucagon mRNA
acttcttctgggaagtctcgc

SEQ ID NO: 37 - DNA, 18 bp, artificial
  oligonucleotide for amplifying pdx1 cDNA
cggacatctccccatacg

SEQ ID NO: 38 - DNA, 18 bp, artificial
  oligonucleotide for amplifying pdx1 cDNA
aaagggagctggacgcgg

SEQ ID NO: 39 - DNA, 22 bp, artificial
  oligonucleotide for amplifying Beta2 (Neurod1) cDNA
actccaagacccagaaactgtc

SEQ ID NO: 40 - DNA, 22 bp, artificial
  oligonucleotide for amplifying Beta2 (Neurod1) cDNA
actggtaggagtagggatgcac

SEQ ID NO: 41 - DNA, 18 bp, artificial
  oligonucleotide for amplifying Glut2 (Slc2a2) cDNA
cattctttggtgggtggc

SEQ ID NO: 42 - DNA, 19 bp, artificial
  oligonucleotide for amplifying Glut2 (Slc2a2) cDNA
cctgagtgtgtttggagcg

SEQ ID NO: 43 - DNA, 22 bp, artificial
  oligonucleotide for amplifying Pc (Pcx) cDNA
gttccgtgtccgaggtgtaaag

SEQ ID NO: 44 - DNA, 22 bp, artificial
  oligonucleotide for amplifying Pc (Pcx) cDNA
cgcagaaggatgtccctgaaac

SEQ ID NO: 45 - DNA, 22 bp, artificial
  oligonucleotide for amplifying Gk (Gck) cDNA
cattgaatcagaggagggcagc

SEQ ID NO: 46 - DNA, 24 bp, artificial
  oligonucleotide for amplifying Gk (Gck) cDNA
tagtggactgggagcatttgtggg

SEQ ID NO: 47 - DNA, 22 bp, artificial
  oligonucleotide for amplifying Gpr40 (Ffar1) cDNA
tattcctggggtgtgtgtgtgg

SEQ ID NO: 48 - DNA, 22 bp, artificial
  oligonucleotide for amplifying Gpr40 (Ffar1) cDNA
ccaagggcagaaagaagagcag

SEQ ID NO: 49 - DNA, 22 bp, artificial
  oligonucleotide for amplifying BiP (Hspa5)
aggacaagaaggaggatgtggg

SEQ ID NO: 50 - DNA, 21 bp, artificial
  oligonucleotide for amplifying BiP (Hspa5)
accgaagggtcattccaagtg

SEQ ID NO: 51 - DNA, 23 bp, artificial
  oligonucleotide for amplifying Chop (Ddit3) cDNA
ttcactactcttgaccctgcgtc

SEQ ID NO: 52 - DNA, 24 bp, artificial
  oligonucleotide for amplifying Chop (Ddit3) cDNA
cactgaccactctgtttccgtttc

SEQ ID NO: 53 - DNA, 24 bp, artificial
  oligonucleotide for amplifying Ho-1 (Hmox1) cDNA
cactgaccactctgtttccgtttc

SEQ ID NO: 54 - DNA, 22 bp, artificial
  oligonucleotide for amplifying Ho-1 (Hmox1) cDNA
gttcgggaaggtaaaaaaagcc

(19) United States
(12) Patent Application Publication
(10) Pub. No.: US 2013/0171158 A1
(43) Pub. Date: Jul. 4, 2013

(54) TREATMENT OF ABNORMALITIES OF GLUCOSE METABOLISM WITH AN ANTAGONIST OF INHIBITOR OF DIFFERENTIATION 1

(71) Applicants: Mia Akerfeldt, Waverly (AU); Ross Laybutt, Killara (AU)

(72) Inventors: Mia Akerfeldt, Waverly (AU); Ross Laybutt, Killara (AU)

(73) Assignee: GARVAN INSTITUTE OF MEDICAL RESEARCH, Darlinghurst, New South Wales (AU)

(21) Appl. No.: 13/806,515

(22) PCT Filed: Jun. 29, 2011
(86) PCT No.: PCT/AU2011/000806

§ 371 Date: Mar. 18, 2013

(30) Foreign Application Priority Data
Jun. 30, 2010 (AU) 2010902914
Apr. 21, 2011 (AU) 2011901521

(51) Int. Cl. A61K 38/17; A61K 31/165; A61K 31/495; A61K 31/4725; A61K 31/519; A61K 31/40; A61K 31/554; A61K 31/445; A61K 31/381; A61K 31/36; A61K 31/366; A61K 31/4162; A61K 31/496; A61K 31/5377; A61K 31/713

(52) U.S. Cl. CPC A61K 38/1709; A61K 31/713; A61K 31/165; A61K 31/495; A61K 31/4725; A61K 31/519; A61K 31/40; A61K 31/554; A61K 31/445; A61K 31/381; A61K 31/36; A61K 31/366; A61K 31/4162; A61K 31/496; A61K 31/5377
USPC 424/139.1; 514/44 A; 514/6.8; 514/615; 514/255.03; 514/309; 514/267; 514/423; 514/211.04; 514/330; 514/447; 514/466; 514/456; 514/407; 514/254.11; 514/443; 514/235.5; 514/733; 435/7.92

(57) ABSTRACT

A method of treating or preventing an abnormality of glucose metabolism in a subject, the method comprising administering an antagonist of Inhibitor of Differentiation 1 (Id1) to the subject.

### FIELD

The present disclosure relates to methods of treating, preventing, diagnosing or prognosing an abnormality of glucose metabolism.

### BACKGROUND

Type 2 diabetes is a serious health concern, particularly in more developed societies that ingest foodstuffs high in sugars and/or fats. The disease is associated with blindness, heart disease, stroke, kidney disease, hearing loss, gangrene and impotence. Type 2 diabetes and its complications are leading causes of premature death in the Western world. There are an estimated 23.6 million people in the United States (7.8% of the population) with diabetes with 17.9 million being diagnosed, 90% of whom suffer from type 2 diabetes. With prevalence rates doubling between 1990 and 2005, the Center for Disease Control (CDC) in USA has characterized the increase as an epidemic. Traditionally considered a disease of adults, type 2 diabetes is increasingly diagnosed in children in parallel to rising obesity rates due to alterations in dietary patterns as well as in life styles during childhood

Generally, type 2 diabetes adversely affects the way the body converts or utilizes ingested sugars and starches into glucose. Despite the presence of insulin resistance at the level of peripheral tissues and the liver, the majority of overweight and obese individuals do not develop diabetes because their pancreatic β-cells adequately respond and prevent overt hyperglycaemia through increased insulin secretion. This is known as β-cell compensation. Those who progress to type 2 diabetes do so because insulin secretion cannot match insulin demand. In this regard, type 2 diabetes is associated with a progressive decline in β-cell function, which is manifest primarily as a selective loss of glucose-stimulated insulin secretion (GSIS). There is now good evidence for reduced β-cell mass linked with increased rates of β-cell apoptosis in people suffering from type 2 diabetes relative to weight-matched subjects without diabetes.

In most type 2 diabetes subjects, the metabolic entry of glucose into various “peripheral” tissues is reduced and there is increased liberation of glucose into the circulation from the liver. Thus, there is an excess of extracellular glucose and a deficiency of intracellular glucose. Elevated blood lipids and lipoproteins are a further common complication of diabetes. The cumulative effect of these diabetes-associated abnormalities is severe damage to blood vessels and nerves.

Many available treatments for type 2 diabetes, some of which have not changed substantially in many years, have recognized limitations. For example, while physical exercise and reductions in dietary intake of fat, high glycemic carbohydrates, and calories can dramatically improve the diabetic condition, compliance with this treatment is very poor because of well-entrenched sedentary lifestyles and excess food consumption, especially of foods containing high amounts of saturated fat.

Conventional drug-based treatments for type 2 diabetes are very limited, and focus on attempting to control blood glucose levels to minimize or delay complications. Current treatments target either insulin resistance (metformin, thiazolidinediones (“TZDs”)), or insulin release from the β-cells (sulphonylureas, exanatide). Sulphonylureas, and other compounds that act by depolarizing the β-cell, have the side effect of hypoglycemia since they cause insulin secretion independent of circulating glucose levels. Other side effects of current therapies include weight gain, loss in responsiveness to therapy over time, gastrointestinal problems, and edema.

One currently approved drug, Januvia (sitagliptin, a dipeptidyl peptidase IV (DPPIV) inhibitor) increases blood levels of incretin hormones (e.g., glucagon-like peptide (GLP)-1), which can increase insulin secretion, reduce glucagon secretion and have other less well characterized effects. However, Januvia and other dipeptidyl peptidase IV (DPPIV) inhibitors may also influence the tissue levels of other hormones and peptides, and the long-term consequences of this broader effect have not been fully investigated. For example, DPPIV is a tumor suppressor, and inhibition of this enzyme may increase the risk of some cancers, e.g., non-small cell lung cancer. Moreover, this compound does not address problems associated with insulin resistance.

The use of clinically available agents that increase intracellular availability of GLP-1, such as orally active dipeptidyl peptidase-4 (DPPIV) inhibitors or injectable GLP-1 analogs, are also limited as a result of relatively short half-life of these agents. This means that they require frequent administration.

It is clear from the foregoing that there is a need in the art for a method to treat or prevent or delay the onset or progression of abnormalities of glucose metabolism, e.g., type 2 diabetes.

### SUMMARY

The present inventors have now determined that the Inhibitor of Differentiation (otherwise known as Inhibitor of DNA Binding) (Id)-1 protein is upregulated in subjects suffering from an abnormality of glucose metabolism, and that this protein is causative of abnormalities of glucose metabolism. For example, the present inventors have now shown that forced overexpression of Id1 in a pancreatic β cell line reduces GSIS. The inventors have also now demonstrated that antagonizing Id-1 expression and/or activity in an accepted mouse model of human type 2 diabetes, reduces and/or prevents glucose intolerance, increases insulin levels and improves GSIS. These effects are observed in subjects' consuming a high-fat diet or a regular diet.

Moreover, in a pancreatic β cell line inhibiting Id1 expression and/or activity reduced glucose stimulated proliferation and increased GSIS. These findings by the inventors provide the basis for methods for treating abnormalities of glucose metabolism.

The inventors have also shown that inhibiting Id1 prevents loss of expression of genes associated with pancreatic β cells and reduces the level of expression of stress response genes in a cell model of a glucose metabolism disorder.

Accordingly, one example of the present disclosure provides a method of treating or preventing an abnormality of glucose metabolism in a subject, the method comprising administering an antagonist of Id1 to the subject, such that the abnormality is treated or prevented.

Each example of the disclosure described herein in relation to treating or preventing an abnormality of glucose metabolism shall be taken to apply mutatis mutandis to increasing or improving insulin secretion, for example GSIS.

In one example, the subject suffers from a condition selected from the group consisting of type 1 diabetes, type 2 diabetes, hyperglycaemia, hyperinsulinemia, insulin resistance, glucose intolerance and combinations thereof. For example, the subject suffers from type 2 diabetes.

The present disclosure contemplates various antagonists of Id1, such as, small molecules, nucleic acids, antibodies or peptides.

In one example, the antagonist binds to Id1.

In one example, the antagonist is a peptide comprising a sequence set forth in any one or more of SEQ ID NOs: 5-16 or 28, or a cell expressing same or a nucleic acid encoding same.

In another example, the antagonist is a small molecule. For example, the antagonist comprises a structure set forth in Formula I or a derivative or salt thereof:

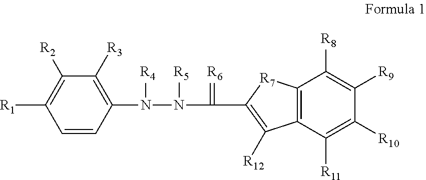

wherein R₁ is a substituted or unsubstituted lower hydrocarbon independently selected from the group consisting of alkyl, alkenyl, alkanoyl, alkynyl, aryl, aroyl, aralkyl, alkylamino, aryloxy, hydrogen, carboxyl, nitro, thioalkoxy, thioaryloxy, thiol, cycloalkenyl cycloalkyl, heterocycloalkyl, heteroaryl, aralkyl, amino acid, peptide, dye, fluorophore, carbohydrate or polypeptide; R₂ and R₃ are independently, collectively, or in any combination selected from hydrogen, hydroxyl, sulfyhydryl, fluorine, methyl, ethyl, propyl, benzyl, 2-bromovinyl amino, hydroxymethyl, methoxy, halogen, pseudohalogen, cyano, carboxyl, nitro, thioalkoxy, thioaryloxy, thiol, substituted or unsubstituted lower hydrocarbons containing 1 to 20 carbons, alkoxycarconyl, allkoxycarbonylamino, amino, amino acid, aminocarbonyl, aminocarbonyloxy, aralkyl, aryloxy, carboxyl, cycloalkenyl, alkyl, cycloalkyl, heterocycloalkyl, aryl, heteroaryl, aralkyl, amino acid, peptide, dye, fluorophore, carbohydrate or polypeptide; R₄ and R₅ are independently, collectively, or in any combination an acyl, or a substituted or unsubstituted lower hydrocarbon independently selected from the group consisting of alkyl, alkenyl, alkanoyl, aryl, aroyl, aralkyl or alkylamino; R₆ is oxygen, sulfur or nitrogen; R₇ is sulfur, nitrogen, oxygen or carbon; R₈, R₉, R₁₀, R₁₁ and Ri₂ are independently, collectively, or in any combination selected from the group consisting of hydrogen, hydroxyl, sulfyhydryl, fluorine, methyl, ethyl, propyl, benzyl, 2-bromovinyl amino, hydroxymethyl, methoxy, halogen, pseudohalogen, cyano and a substituted and unsubstituted lower hydrocarbon containing 1 to 20 carbons.

In another example, the antagonist comprises a structure set forth in Formula II or a derivative or salt thereof:

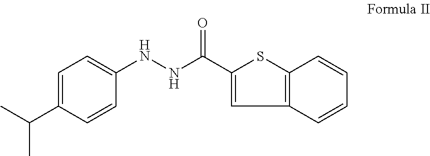 [SMILES: CC(C)c1ccc(NNC(=O)c2cc3ccccc3s2)cc1]

In a further example, the antagonist comprises a structure set forth in Formula III or a derivative or salt thereof:

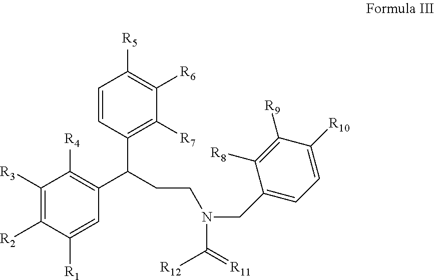

wherein, R₁, R₂, R₃, R₄, R₅, R₆, R₈, R₉, and R₁₀ may independently, collectively, or in any combination be selected from the group consisting of hydrogen, hydroxyl, sulfyhydryl, fluorine, methyl, ethyl, propyl, benzyl, 2-bromovinyl amino, hydroxymethyl, methoxy, halogen, pseudohalogen, cyano, carboxyl, nitro, thioalkoxy, thioaryloxy, thiol, substituted or unsubstituted lower hydrocarbon containing 1 to 20 carbons, alkoxycarconyl, allkoxycarbonylamino, amino, amino acid, aminocarbonyl, aminocarbonyloxy, aralkyl, aryloxy, carboxyl, cycloalkenyl, substituted or unsubstituted alkyl, substituted or unsubstituted cycloalkyl, substituted or unsubstituted heterocycloalkyl, substituted or unsubstituted aryl, substituted or unsubstituted heteroaryl, substituted or unsubstituted aralkyl, amino acid, peptide, dye, fluorophore, carbohydrate or polypeptide; R₇ is hydrogen, hydroxyl, benzoyl; substituted benzoyl or hydroxyl substituted with unsubstituted lower hydrocarbon containing 1 to 20 carbons; R₁₁ is oxygen, or another heteroatom such as sulfur or nitrogen; and R₁₂ is a substituted or unsubstituted lower hydrocarbon independently selected from the group consisting of alkyl, alkenyl, alkanoyl, alkynyl, aryl, aroyl, aralkyl, alkylamino, aryloxy, hydrogen, carboxyl, nitro, thioalkoxy, thioaryloxy, thiol, cycloalkenyl substituted or unsubstituted heteroatom, substituted or unsubstituted alkyl, substituted or unsubstituted cycloalkyl, substituted or unsubstituted heterocycloalkyl, substituted or unsubstituted aryl, substituted or unsubstituted heteroaryl, substituted or unsubstituted aralkyl, amino acid, peptide, dye, fluorophore, carbohydrate or polypeptide.

In a still further example, the antagonist comprises a structure set forth in Formula IV or a derivative or salt thereof:

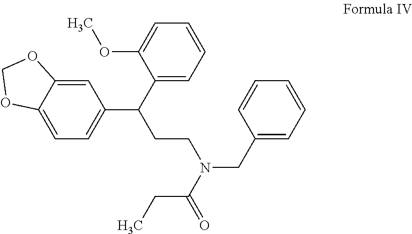 [SMILES: CCC(=O)N(CCC(c1ccc2c(c1)OCO2)c1ccccc1OC)Cc1ccccc1]

In a further example, the antagonist is a small molecule selected from the group consisting of: 1-(4-methoxybenzyl)-4-(2,4,5-trimethoxybenzyl)piperazine; N′-acetyl-N′-(3-chloro-4-methylphenyl)-2-hydroxy-2,2-diphenylacetohydrazide; 6,7-dimethoxy-2-methyl-1-oxo-3-pyridin-3-yl-N-[3-(trifluoromethyl)phenyl]-1,2,3,4-tetrahydroisoquinoline-4-carboxamide; 2-[3-(3,4-dimethylphenyl)-2,4-dioxo-3,4,6,7,8,9-hexahydro-2H-cyclohepta[4,5]thieno[2,3-d]pyrimidin-1 (5H)-yl]-N-(2-methoxyethyl)acetamide; 4-acetyl-N,N-dibutyl-3,5-dimethyl-1H-pyrrole-2-carboxamide; N-(4-isopropylphenyl)-2-[8-morpholin-4-ylcarbonyl)-11-oxodibenzo[b,f][1,4]thiazepin-10(11H)-yl]acetamide; ethyl 1-(3-[(2,5-dimethyloxyphenyl)amino]-3-oxopropyl)-4-piperidinecarboxylate hydrochloride; ethyl 2-amino-4-methyl-5-({[2-(trifluoromethyl)phenyl]amino}carbonyl)-3-thiophenecarboxylate; N-[3-) 1,3-benzodioxol-5-yl)-3-(3-methoxyphenyl)propyl]-N-benzylpropanamide; methyl{7-[(3-methoxybenzyl)oxy]-4,8-dimethyl-2-oxo-2H-chromen-3-yl}acetate; ethyl 3-hydroxy-5-methyl-6-oxo-1-phenyl-1,6-dihydropyrano[2,3-c]pyrazole-4-carboxylate; 1-(1,3-benzodioxol-5-ylmethyl)-4-[(4-chlorobenzyl)sulfonyl]piperazine; N′-(4-isopropylphenyl)-1-benzothiophene-2-carbohydrazide; 5-)4-bromophenyl)-3-hydroxy-4-(3-methyl-4-propoxybenzoyl)-1-[2-(4-morpholinyl)ethyl]-1,5-dihydro-2H-pyrrol-2-one; 5-(4-fluorophenyl)-3-hydroxy-4-(4-isobutoxy-3-methyl)-1-[2-(4-morpholinyl]-1,5-dihydro-2H-pyrrol-2one; and 5-(3-bromophenyl)-4-(3-fluoro-4-methoxybenzoyl)-3-hydroxy-1-[3-(4-morpholinyl)propyl]-1,5-dihydro-2H-pyrrol-2-one.

In another example, the antagonist reduces or prevents expression of Id1. For example, the antagonist binds to an Id1 encoding nucleic acid and reduces Id1 expression. In one example, the antagonist is a nucleic acid which is an antisense, a siRNA, a RNAi, a microRNA, a DNAzyme or a RNAzyme. Exemplary nucleic acid antagonists include those comprising a sequence set forth in any one of SEQ ID NOs: 17-21.

In another example, the antagonist is a cannabidiol.

In some examples, an antagonist is linked to a compound that targets a pancreas in a subject. Exemplary pancreatic targeting compounds include peptides, e.g., a peptide comprising a sequence set forth in any one or more of SEQ ID NOs: 22-25. In the case of a peptide antagonist, the antagonist can be a fusion protein comprising the peptide antagonist and a pancreatic targeting peptide.

In some examples, an Id1 antagonist is linked to a compound that facilitates cellular uptake. For example, the antagonist is linked to a protein transduction domain, e.g., a peptide comprising a sequence set forth in SEQ ID NO: 26 or 27. In the case of a peptide antagonist, the antagonist can be a fusion protein comprising the peptide antagonist and a protein transduction domain, optionally together with pancreatic targeting peptide.

The antagonist of the disclosure can be administered to the subject in any suitable manner. In one example, the antagonist is administered to the pancreas of the subject or to a blood vessel supplying a pancreas of a subject.

In one example of a method of the disclosure, the antagonist is administered in the form of a pharmaceutical composition, e.g., additionally comprising a pharmaceutically acceptable carrier.

In one example, the subject suffers from an abnormality characterized by increased Id1 expression in the pancreas or a cell or tissue thereof.

In one example, the method additionally comprises detecting the level of expression of Id1 in the pancreas or a cell or tissue thereof of the subject.

In one example, the subject is in need of treatment. For example, the subject suffers from an abnormality of glucose metabolism. For example, the subject suffers from type 2 diabetes, hyperglycaemia, hyperinsulinemia, insulin resistance, glucose intolerance and combinations thereof. For example, the subject suffers from type 2 diabetes.

In a further example, the subject is at risk of developing an abnormality of glucose metabolism. For example, the subject is obese and/or suffers from insulin resistance and/or suffers from pancreatitis and/or has a family history of an abnormality of glucose metabolism.

In one example, the subject consumes a high calorie diet. For example, the subject consumes more than about 3500-4000 calories per day, for example more than 4000 calories per day.

In one example, the subject consumes a high fat diet. For example, more than about 30% or 40% or 50% of calories consumed by the subject is obtained from fat.

In one example, the subject does not consume a high fat diet.

The present disclosure also provides an antagonist of Id1 for use in the treatment of an abnormality of glucose metabolism.

The present disclosure also provides for use of an antagonist of Id1 in the manufacture of a medicament for the treatment of an abnormality of glucose metabolism.

Suitable antagonists and/or abnormalities are described herein and are to be taken to apply mutatis mutandis to the previous two examples of the disclosure.

As discussed above, the inventors have determined that Id1 is overexpressed in an abnormality of glucose metabolism and is causative of this condition. Accordingly, the present disclosure also provides a method of diagnosing or prognosing an abnormality of glucose metabolism a subject, the method comprising detecting the level of expression of Id1 in a sample from the subject, wherein an increased level of Id1 is diagnostic or prognostic of an abnormality of glucose metabolism in the subject.

In one example, the method comprises:

(i) detecting the level of expression of Id1 in the sample from the subject; and
(ii) comparing the level of expression of Id1 to the level of expression of Id1 in a control sample,
wherein an increased level of Id1 is diagnostic or prognostic of an abnormality of glucose metabolism in the subject.

In one example, the sample from the subject comprises a pancreatic cell or comprises pancreatic tissue.

### BRIEF DESCRIPTION OF THE DRAWINGS

FIG. 1A is a graphical representation showing blood glucose levels during an intraperitoneal glucose tolerance test (i.p. GTT) of wild-type (white triangles, n=19) and Id1^{-/-} (black triangles, n=22) mice fed a chow diet and wild-type (white squares, n=12) and Id1^{+/-} (black squares, n=6) mice fed a 6 week high-fat diet. ANOVA: p<0.0001 for effect of diet in wild-type mice, p<0.05 for effect of diet in Id1^{-/-} mice, p<0.0001 for effect of Id1 deletion in chow-fed mice, p<0.0001 for effect of Id1 deletion in fat-fed mice.

FIG. 1B is a graphical representation showing area under the curve (AUC) of blood glucose levels during the i.p. GTT described in respect of FIG. 1A. ***p<0.001 effect of fat diet in wild-type mice, †p<0.05 effect of Id1 deletion in chow-fed mice, †††p<0.001 for effect of Id1 deletion in fat-fed mice.

FIG. 1C is a graphical representation showing insulin levels during i.p. GTT of wild-type (n=14) and Id1^{-/-} (n=17) mice fed a chow diet and wild-type (n=11) and Id1^{-/-} (n=6) mice fed a high-fat diet. ANOVA: p<0.01 for effect of fat diet in wild-type mice, p<0.0001 for effect of fat diet in Id1^{-/-} mice, p<0.05 for effect of Id1 deletion in chow-fed mice, p<0.001 for effect of Id1 deletion in fat-fed mice.

FIG. 1D is a graphical representation showing AUC of insulin levels during i.p. GTT as described in respect of FIG. 1C. **p<0.01 for effect of fat diet in wild-type mice, †p<0.05 for effect of Id1 deletion in chow-fed mice, †††p<0.001 for effect of Id1 deletion in fat-fed mice.

FIG. 2 is a graphical representation showing the effect of Id1 deletion on glucose tolerance in mice fed a standard chow or a high-fat diet for 18 weeks. Blood glucose levels during an intraperitoneal glucose tolerance test (i.p. GTT) of wild-type (white triangles, n=6) and Id1^{-/-} (black triangles, n=10) mice fed a chow diet and wild-type (white squares, n=7) and Id1−/− (black squares, n=6) mice fed a high-fat diet for 18 weeks. ANOVA: p<0.05 for effect of diet in wild-type mice, p<0.05 for effect of Id1 deletion in chow-fed mice, p<0.05 for effect of Id1 deletion in fat-fed mice.

FIG. 3 is a graphical representation showing the effect of Id3 deletion on glucose tolerance in mice fed a standard chow or a high-fat diet for 6 weeks. Blood glucose levels during an intraperitoneal glucose tolerance test (i.p. GTT) of wild-type (white triangles, n=6) and Id3^{-/-} (black triangles, n=7) mice fed a chow diet and wild-type (white squares, n=11) and Id3^{-/-} (black squares, n=10) mice fed a high-fat diet for 6 weeks. ANOVA: p<0.01 for effect of diet in wild-type and Id3^{-/-} mice.

FIG. 4A is a graphical representation showing blood glucose levels during an intraperitoneal insulin tolerance test (i.p. ITT) of wild-type (white triangles, n=5) and Id1^{-/-} (black triangles, n=4) mice fed a chow diet and wild-type (white squares, n=8) and Id1^{-/-} (black squares, n=6) mice fed a high-fat diet. ANOVA: p<0.05 for effect of fat diet in wild-type and Id1^{-/-} mice.

FIG. 4B is a graphical representation showing AUC 0-30 min of blood glucose levels during i.p. ITT described in respect of FIG. 4A. *p<0.05 for effect of diet in wild-type and Id1^{-/-} mice.

FIG. 5A is a graphical representation showing β-cell mass of wild-type (n=3) and Id1^{-/-} (n=4) mice fed a chow diet and wild-type (n=4) and Id1^{-/-} (n=5) mice fed a high-fat diet. ANOVA: p<0.05 for effect of fat diet in wild-type and Id1^{-/-} mice.

FIG. 5B is a graphical representation showing the number of islets per area of pancreas of wild-type (n=3) and Id1^{-/-} (n=4) mice fed a chow diet and wild-type (n=4) and Id1^{-/-} (n=5) mice fed a high-fat diet. ANOVA: p<0.01 for effect of fat diet in wild-type and Id1^{-/-} mice.

FIG. 6 is a graphical representation showing the effects of Id1 deletion on GSIS in isolated islets. Batches of islets isolated from wild-type mice fed a chow (striped bars, n=6) or a high-fat (white bars, n=7) diet and Id1−/− mice fed a chow (hatched bars, n=7) or a high-fat (black bars, n=5) diet were incubated at low (2.8 mM) or high glucose (16.7 mM) for 1 h. Insulin was measured in an aliquot of the media by radioimmunoassay. *p<0.05 for effect of fat diet in wild-type mouse islets at low glucose, **p<0.01 for effect of fat diet in Id1−/− mouse islets at low glucose, †p<0.05 for effect of genotype in fat-fed mouse islets at low and high glucose, ††p<0.01 for effect of genotype in chow-fed mouse islets at high glucose.

FIG. 7A is a graphical representation showing expression levels of Id1 in islets of wild-type mice fed a chow (striped bars, n=5) or a high-fat (white bars, n=6) diet. *p<0.05 for effect of fat diet.

FIG. 7B is a graphical representation showing expression levels of the genes indicated in wild-type mice fed a chow (striped bars, n=5-6) or a high-fat (white bars, n=5-7) diet and Id1^{-/-} mice fed a chow (hatched bars, n=5-7) or a high-fat (black bars, n=4-7) diet. *p<0.05 for effect of fat diet in wild-type mice, †p<0.05, ††p<0.01 for effect of genotype in chow-fed mice.

FIG. 8 includes a copy of a photograph and a graphical representation showing overexpression of Id1 in MIN6 β-cells reduces GSIS. P<0.05, high glucose response in Id1- versus GFP-expressing MIN6 cells.

FIG. 9A is a graphical representation showing expression of Id1 in MIN6 cells treated as indicated. n=5 separate experiments in each group. **p<0.01 for palmitate effect in control siRNA-transfected cells, *p<0.05 for palmitate effect in Id1 siRNA-transfected cells, †††p<0.001 for effect of Id1 siRNA in BSA- and palmitate-treated cells.

FIG. 9B is a graphical representation showing insulin secretion in MIN6 cells. Results are presented as percentage of insulin secretion in control siRNA-transfected BSA pre-treated cells incubated with 25 mM glucose After pre-treatment, cells were incubated in medium containing 2.8 or 25 mM glucose for 1 h. Medium was taken to determine levels of insulin secretion in control siRNA-transfected cells pre-treated with BSA (striped bars) or BSA-coupled palmitate (white bars) and in Id1 siRNA-transfected cells pre-treated with BSA (hatched bars) or BSA-coupled palmitate (black bars). n=4 separate experiments in each group. *p<0.05 for effect of palmitate pre-treatment in control siRNA transfected cells at 25 mM glucose. †p<0.05 for effect of Id1 siRNA in palmitate pre-treated cells at 25 mM glucose.

FIG. 9C is a graphical representation showing total insulin content in cell lysates of cells described in respect of FIG. 9B. *p<0.05 for effect of palmitate pre-treatment in control siRNA-transfected cells, **p<0.01 for effect of palmitate pre-treatment in Id1 siRNA-transfected cells.

FIG. 9D is a graphical representation showing ratio of insulin secretion to total insulin content of cells described in respect of FIG. 9B. Expressed as a percentage of ratios in control siRNA-transfected BSA pretreated cells incubated with 25 mM glucose. †p<0.05 for effect of Id1 siRNA in palmitate pretreated cells at 25 mM glucose.

FIG. 10 is a graphical representation showing relative gene expression levels in MIN6 cells. MIN6 cells transfected with Id1 ONTARGETplus SMARTpool siRNA or Negative Control Non-Targeting siRNA were treated with either 0.92% BSA alone or 0.92% BSA coupled to 0.4 mM palmitate for 48 h. Total RNA was extracted, reverse-transcribed and relative expression of the genes indicated determined by RT-PCR for control siRNA-transfected cells treated with BSA (striped bars) or BSA-coupled palmitate (white bars) and Id1 siRNA-transfected cells treated with BSA (hatched bars) or BSA coupled palmitate (black bars). Results are expressed as a percentage of mRNA levels in control siRNA-transfected cells treated with BSA. n=4-7 in each group. *p<0.05 for effect of palmitate treatment in control siRNA- and Id1 siRNA-transfected cells. †p<0.05, ††p<0.01 for effect of Id1 siRNA in palmitate-treated cells.

FIG. 11 is a graphical represenatation showing that an inhibitor of Id1 expression (Cannabidiol) protects MIN6 beta cells against lipid (palmitate)-induced insulin secretory dysfunction. Cells were treated with either 0.92% BSA or 0.92% BSA coupled to 0.4 mM palmitate for 48 h, in combination with the absence or presence of 10 μM Cannabidiol. Insulin secretion assay was performed. After 30-min preincubation in KRB medium containing 2.8 mM glucose, the cells were incubated in KRB medium containing either 2.8 mM glucose (clear bars) or 25 mM glucose (dark bars) for 1 h. Medium was taken to determine levels of insulin secretion. Insulin secretion expressed as fold change compared with Control cells incubated with 25 mM glucose. Results are means±SE.

### DETAILED DESCRIPTION

### General

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

Each example of the disclosure described herein is to be applied mutatis mutandis to each and every other example unless specifically stated otherwise.

Those skilled in the art will appreciate that the disclosure described herein is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations or any two or more of said steps or features.

The present disclosure is not to be limited in scope by the specific examples described herein, which are intended for the purpose of exemplification only. Functionally-equivalent products, compositions and methods are clearly within the scope of the disclosure, as described herein.

The present disclosure is performed without undue experimentation using, unless otherwise indicated, conventional techniques of molecular biology, microbiology, virology, recombinant DNA technology, peptide synthesis in solution, solid phase peptide synthesis, and immunology. Such procedures are described, for example, in Sambrook, Fritsch & Maniatis, Molecular Cloning: A Laboratory Manual, Cold Spring Harbor Laboratories, New York, Second Edition (1989), whole of Vols I, II, and III; DNA Cloning: A Practical Approach, Vols. I and II (D. N. Glover, ed., 1985), IRL Press, Oxford, whole of text; Oligonucleotide Synthesis: A Practical Approach (M. J. Gait, ed, 1984) IRL Press, Oxford, whole of text, and particularly the papers therein by Gait, pp 1-22; Atkinson et al, pp 35-81; Sproat et al, pp 83-115; and Wu et al, pp 135-151; 4. Nucleic Acid Hybridization: A Practical Approach (B. D. Hames & S. J. Higgins, eds., 1985) IRL Press, Oxford, whole of text; Immobilized Cells and Enzymes: A Practical Approach (1986) IRL Press, Oxford, whole of text; Perbal, B., A Practical Guide to Molecular Cloning (1984); Methods In Enzymology (S. Colowick and N. Kaplan, eds., Academic Press, Inc.), whole of series; J. F. Ramalho Ortigao, “The Chemistry of Peptide Synthesis” In: Knowledge database of Access to Virtual Laboratory website (Interactiva, Germany); Sakakibara, D., Teichman, J., Lien, E. Land Fenichel, R. L. (1976). Biochem. Biophys. Res. Commun. 73 336-342; Merrifield, R. B. (1963). J. Am. Chem. Soc. 85, 2149-2154; Barany, G. and Merrifield, R. B. (1979) in The Peptides (Gross, E. and Meienhofer, J. eds.), vol. 2, pp. 1-284, Academic Press, New York. 12. Wunsch, E., ed. (1974) Synthese von Peptiden in Houben-Weyls Metoden der Organischen Chemie (Miller, E., ed.), vol. 15, 4th edn., Parts 1 and 2, Thieme, Stuttgart; Bodanszky, M. (1984) Principles of Peptide Synthesis, Springer-Verlag, Heidelberg; Bodanszky, M. & Bodanszky, A. (1984) The Practice of Peptide Synthesis, Springer-Verlag, Heidelberg; Bodanszky, M. (1985) Int. J. Peptide Protein Res. 25, 449-474; Handbook of Experimental Immunology, Vols. I-IV (D. M. Weir and C. C. Blackwell, eds., 1986, Blackwell Scientific Publications); and Animal Cell Culture Practical Approach, Third Edition (John R. W. Masters, ed., 2000), ISBN 0199637970, whole of text.

The term “and/or”, e.g., “X and/or Y” shall be understood to mean either “X and Y” or “X or Y” and shall be taken to provide explicit support for both meanings or for either meaning.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### KEY TO SEQUENCE LISTING

SEQ ID NO: 1 is a nucleotide sequence encoding a human Id1 protein.
SEQ ID NO: 2 is an amino acid sequence of a human Id1 protein.
SEQ ID NO: 3 is a nucleotide sequence encoding a human Id1 protein.
SEQ ID NO: 4 is an amino acid sequence of a human Id1 protein.
SEQ ID NO: 5 is an amino acid sequence of a peptide antagonist of Id1.
SEQ ID NO: 6 is an amino acid sequence of a peptide antagonist of Id1.
SEQ ID NO: 7 is an amino acid sequence of a peptide antagonist of Id1.
SEQ ID NO: 8 is an amino acid sequence of a peptide antagonist of Id1.
SEQ ID NO: 9 is an amino acid sequence of a peptide antagonist of Id1.
SEQ ID NO: 10 is an amino acid sequence of a peptide antagonist of Id1.
SEQ ID NO: 11 is an amino acid sequence of a peptide antagonist of Id1.
SEQ ID NO: 12 is an amino acid sequence of a peptide antagonist of Id1.
SEQ ID NO: 13 is an amino acid sequence of a peptide antagonist of Id1.
SEQ ID NO: 14 is an amino acid sequence of a peptide antagonist of Id1.
SEQ ID NO: 15 is an amino acid sequence of a peptide antagonist of Id1.
SEQ ID NO: 16 is an amino acid sequence of a peptide antagonist of Id1.
SEQ ID NO: 17 is a nucleotide sequence of an antisense oligonucleotide that silences Id1 expression.
SEQ ID NO: 18 is a nucleotide sequence of an antisense oligonucleotide that silences Id1 expression.
SEQ ID NO: 19 is a nucleotide sequence of an antisense oligonucleotide that silences Id1 expression.
SEQ ID NO: 20 is a nucleotide sequence of an antisense oligonucleotide that silences Id1 expression.
SEQ ID NO: 21 is a nucleotide sequence of an antisense oligonucleotide that silences Id1 expression.
SEQ ID NO: 22 is an amino acid sequence of a pancreas targeting peptide.
SEQ ID NO: 23 is an amino acid sequence of a pancreas targeting peptide.
SEQ ID NO: 24 is an amino acid sequence of a pancreatic islet targeting peptide.
SEQ ID NO: 25 is an amino acid sequence of a pancreatic islet targeting peptide.
SEQ ID NO 26: is an amino acid sequence of a HIV-1 TAT protein transduction domain.
SEQ ID NO: 27 is an amino acid sequence of an Antennapedia protein transduction domain.
SEQ ID NO: 28 is an amino acid sequence of a peptide antagonist of Id1.
SEQ ID NO: 29 is a nucleotide sequence of an oligonucleotide for amplifying Cyclophilin A (Ppia).
SEQ ID NO: 30 is a nucleotide sequence of an oligonucleotide for amplifying Cyclophilin A (Ppia)
SEQ ID NO: 31 is a nucleotide sequence of an oligonucleotide for amplifying Id1
SEQ ID NO: 32 is a nucleotide sequence of an oligonucleotide for amplifying Id1
SEQ ID NO: 33 is a nucleotide sequence of an oligonucleotide for amplifying Insulin
SEQ ID NO: 34 is a nucleotide sequence of an oligonucleotide for amplifying Insulin
SEQ ID NO: 35 is a nucleotide sequence of an oligonucleotide for amplifying Glucagon
SEQ ID NO: 36 is a nucleotide sequence of an oligonucleotide for amplifying Glucagon
SEQ ID NO: 37 is a nucleotide sequence of an oligonucleotide for amplifying Pdx1
SEQ ID NO: 38 is a nucleotide sequence of an oligonucleotide for amplifying Pdx1
SEQ ID NO: 39 is a nucleotide sequence of an oligonucleotide for amplifying Beta2 (Neurod1)
SEQ ID NO: 40 is a nucleotide sequence of an oligonucleotide for amplifying Beta2 (Neurod1)
SEQ ID NO: 41 is a nucleotide sequence of an oligonucleotide for amplifying Glut2 (Slc2a2)
SEQ ID NO: 42 is a nucleotide sequence of an oligonucleotide for amplifying Glut2 (Slc2a2)
SEQ ID NO: 43 is a nucleotide sequence of an oligonucleotide for amplifying Pc (Pcx)
SEQ ID NO: 44 is a nucleotide sequence of an oligonucleotide for amplifying Pc (Pcx)
SEQ ID NO: 45 is a nucleotide sequence of an oligonucleotide for amplifying Gk (Gck)
SEQ ID NO: 46 is a nucleotide sequence of an oligonucleotide for amplifying Gk (Gck)
SEQ ID NO: 47 is a nucleotide sequence of an oligonucleotide for amplifying Gpr40 (Ffar1)
SEQ ID NO: 48 is a nucleotide sequence of an oligonucleotide for amplifying Gpr40 (Ffar1)
SEQ ID NO: 49 is a nucleotide sequence of an oligonucleotide for amplifying BiP (Hspa5)
SEQ ID NO: 50 is a nucleotide sequence of an oligonucleotide for amplifying BiP (Hspa5)
SEQ ID NO: 51 is a nucleotide sequence of an oligonucleotide for amplifying Chop (Ddit3) 176
SEQ ID NO: 52 is a nucleotide sequence of an oligonucleotide for amplifying Chop (Ddit3) 176
SEQ ID NO: 53 is a nucleotide sequence of an oligonucleotide for amplifying Ho-1 (Hmox1)
SEQ ID NO: 54 is a nucleotide sequence of an oligonucleotide for amplifying Ho-1 (Hmox1)

### SELECTED DEFINITIONS

As used herein, the term “inhibitor of differentiation 1” or “inhibitor of DNA binding” or “Id1” will be understood to mean a helix-loop-helix (HLH) protein that can form heterodimers with members of the basic HLH family of transcription factors. The encoded protein has no detectable DNA binding activity and therefore can inhibit the DNA binding and transcriptional activation ability of basic HLH proteins with which it interacts. Exemplary sequences of Id1 protein are set forth in NCBI Accession No. 3397. For the purposes of nomenclature only, and not limitation, amino acid sequences of Id1 are set forth in SEQ ID NOs: 2 and 4. Sequences of nucleic acids encoding Id1 are set forth in SEQ ID NOs: 1 and 3.

As used herein, the term “abnormality of glucose metabolism” shall be taken to mean a condition characterised by hyperglycemia, glucose intolerance, insulin resistance, hyperinsulinemia and/or β-islet cell dysfunction. For example, the abnormality of glucose metabolism is type 2 diabetes.

As used herein, the term “antagonist of Id1” shall be taken to mean a compound that reduces, prevents or inhibits the activity of Id1 protein and/or that reduces, prevents or inhibits expression of Id1. For example, the antagonist binds to Id1 or nucleic acid encoding same, i.e., acts directly on Id1 or nucleic acid encoding same. In some examples, the antagonist is specific for Id1. A compound that reduces, prevents or inhibits the activity of Id1 shall be understood to act at the level of the Id1 protein. A compound that reduces, prevents or inhibits expression of Id1, will necessarily reduce the Id1 activity level by virtue of reducing the level of the protein, e.g., in a cell. An activity of Id1 that may be inhibited by the antagonist is its ability to dimerize with a HLH transcription factor, such as, E47.

By “specific for Id1” shall be understood to mean that an antagonist reacts or associates more frequently, more rapidly, with greater duration and/or with greater affinity with Id1 or a cell expressing same than it does with alternative proteins, e.g., other Id proteins (e.g., Id3) or cells. Specific binding does not necessarily require exclusive binding or non-detectable binding to another protein, this is meant by the term “selective binding”. Generally, but not necessarily, reference to binding means specific binding, and each term shall be understood to provide explicit support for the other term.

As used herein, the terms “preventing”, “prevent” or “prevention” in the context of preventing a condition include administering an amount of a protein described herein sufficient to stop or hinder the development of at least one symptom of a specified disease or condition.

As used herein, the terms “treating”, “treat” or “treatment” include administering a therapeutically effective amount of an inhibitor(s) and/or agent(s) described herein sufficient to reduce or eliminate at least one symptom of a specified disease or condition.

As used herein, the term “diagnosis”, and variants thereof such as, but not limited to, “diagnose”, “diagnosed” or “diagnosing” includes any primary diagnosis of a clinical state or diagnosis of recurrent disease.

“Prognosis”, “prognosing” and variants thereof as used herein refer to the likely outcome or course of a disease, including the chance of recovery or recurrence or the outcome of treatment.

As used herein, the term “subject” shall be taken to mean any animal including humans, for example a mammal. Exemplary subjects include but are not limited to humans, primates, livestock (e.g. sheep, cows, horses, donkeys, pigs), companion animals (e.g. dogs, cats), laboratory test animals (e.g. mice, rabbits, rats, guinea pigs, hamsters), captive wild animals (e.g. fox, deer). For example, the mammal is a human or primate. For example, the mammal is a human.

Reference herein to a “sample” should be understood as a reference to any sample derived from a subject such as, but not limited to, a pancreas or part thereof or a body fluid (e.g., blood or synovial fluid or cerebrospinal fluid), cellular material (e.g. tissue aspirate), tissue biopsy specimens or surgical specimens. The “sample” includes extracts and/or derivatives and/or fractions of the sample.

### Antagonists of Id1 Activity

Exemplary antagonists of Id1, antagonize Id1 activity. For example, such antagonists bind to Id1 and antagonize this protein.

### Small Molecule Antagonists

In one example, an antagonist of Id1 activity is a small molecule. Exemplary small molecules are described herein, e.g., in Formulae I-V. Such small molecule antagonists are also described, for example, in WO01/66116 and WO2009/051801.

The skilled artisan will understand that any of the substitutions contemplated to the compounds described herein will maintain the Id1 antagonizing activity of the compound, together with an ability to treat or prevent an abnormality of glucose metabolism.

The present disclosure contemplates a pharmaceutically acceptable active salt of a compound described herein according to any example of the disclosure, as well as active isomers, enantiomers, polymorphs, solvates, hydrates and/or prodrugs of those compounds.

With regard to a compound comprising a structure set forth in Formula 1, when more than one R group is present, the R group may be selected from any of the groups recited herein so as to be the same or different. In additional examples, two or more R groups may be joined together. In some examples, R₂ and R₃ may be members of a 5, or 6, member exocyclic ring structure. In other examples, R₃ and R₄ may be members of a 5, or 6, member exocyclic ring structure. In further examples, R₅ and R₆ may be members of a 5 or 6 member exocyclic ring structure. In additional examples, R₁₁ and R₁₂ may be members of a 5 or 6 member exocyclic ring structure. In some examples, if R₇ is nitrogen, R₆ and R₇ may be members of a 5 or 6 member exocyclic ring structure. In other examples, R₆ and R₁₂ may be members of a 5 or 6 member exocyclic ring structure.

One having ordinary skill in the art will recognize that the maximum number of heteroatoms in a stable, chemically feasible heterocyclic ring, whether it is aromatic or non-aromatic, is determined by the size of the ring, the degree of unsaturation and the valence of the heteroatoms. In general, a heterocyclic ring may have one to four heteroatoms so long as the heteroaromatic ring is chemically feasible and stable.

In examples of the disclosure, an antagonist of Id1 comprising a structure set forth in Formula I is be N′-(4-isopropylphenyl)-1-benzothiophene-2-carbohydrazide as shown in Formula II or a derivative thereof.

With regard to a compound comprising a structure set forth in Formula III, when more than one R group is present, the R group may be selected from any of the stated groups so as to be the same or different. In additional examples, two or more R groups may be joined together. In some examples, R₄ may become a member of a 5 or 6 member ring structure with neighboring rings.

In some examples, a compound comprising a structure set forth in Formula III may be N-[3-(1,3-benzodioxol-5-yl)-3-(2-methoxyphenyl) propyl]-N-benzylpropanamide as shown in Formula IV, or a derivative thereof.

In some examples of the disclosure, a an antagonist of Id1, e.g., N-[3-(1,3-benzodioxol-5-yl)-3-(2-methoxyphenyl)propyl]-N-benzylpropanamide may be part of a racemic mixture. This mixture can be resolved using standard methods and either enantiomer used as a therapeutic. The addition of another asymmetric center in the molecule would introduce the possibility of diastereomers. Useful anti-Id related compounds and derivatives of Formulas I, II, III or IV within the formulations and methods herein include, but are not limited to, other pharmaceutically acceptable active salts of said compounds, as well as active isomers, enantiomers, polymorphs, solvates, hydrates, and/or prodrugs of said compounds.

“Stereoisomer” as it relates to a given compound is understood in the art, and refers to another compound having the same molecular formula, wherein the atoms making up the other compound differ in the way they are oriented in space, but wherein the atoms in the other compound are like the atoms in the given compound with respect to which atoms are joined to which other atoms (e.g. an enantiomer, a diastereomer, or a geometric isomer). See for example, Morrison and Boyd, Organic Chemistry, 1983, 4th ed., Allyn and Bacon, Inc., Boston, Mass., p. 123.

“Substituted” as used herein refers to a group in which one or more hydrogen atoms are each independently replaced with the same or different substituent(s). “Substituted” groups particularly refer to groups having 1 or more substituents, for instance from 1 to 5 substituents, and particularly from 1 to 3 substituents, selected from the group consisting of acyl, acylamino, acyloxy, alkoxy, substituted alkoxy, alkoxycarbonyl, alkoxycarbonylamino, amino, substituted amino, aminocarbonyl, aminocarbonylamino, aminocarbonyloxy, aryl, aryloxy, azido, carboxyl, cyano, cycloalkyl, substituted cycloalkyl, halogen, hydroxyl, keto, nitro, thioalkoxy, substituted thioalkoxy, thioaryloxy, thioketo, thiol, alkyl-S(O)—, aryl-S(O)—, alkyl-S(O)₂— and aryl-S(O)₂. Typical substituents include, but are not limited to, —X, —O—, =0, —OR₈, —SR₈, —S—, —S, —NR₈R₉, ═NR₈, —CX₃, —CF₃, —CN, —OCN, —SCN, —NO, —NO₂, ═N₂, —N₃, —S(O)₂O—, —S(O)₂OH, —S(O)₂R₈, —OS(O₂)O—, —OS(O)₂R₈, —P(O)(O—)₂, —P(O)(OR₈)(O—), —OP(O)(OR₈)(OR₉), —C(O)R₈, —C(S)R₈, —C(O)OR₈, —C(O)NR₈R₉, —C(O)O—, —C(S)OR₈, —NR₁₀C(O)NR₈R₉, —NR₁₀C(S)NR₈R₉, —NR₁₁C(NR₁₀)NR₈R₉ and —C(NRi₀)NR₈R₉, where each X is independently a halogen.

In one example, the antagonist is not tetracycline or a derivative thereof.

Small molecule compounds according to the present disclosure can be produced using standard techniques or purchased from a commercial source. For example, Chemdiv Inc produces 1-(4-methoxybenzyl)-4-(2,4,5-trimethoxybenzyl)piperazine; N′-acetyl-N′-(3-chloro-4-methylphenyl)-2-hydroxy-2,2-diphenyl acetohydrazide; 6,7-dimethoxy-2-methyl-1-oxo-3-pyridin-3-yl-N-[3-(trifluoromethyl)phenyl]-1,2,3,4-tetrahydroisoquinoline-4-carboxamide; 2-[3-(3,4-dimethylphenyl)-2,4-dioxo-3,4,6,7,8,9-hexahydro-2H-cyclohepta[4,5]thieno[2,3-d]pyrimidin-1 (5H)-yl]-N-(2-methoxyethyl)acetamide; 4-acetyl-N,N-dibutyl-3,5-dimethyl-1H-pyrrole-2-carboxamide; N-(4-isopropylphenyl)-2-[8-morpholin-4-ylcarbonyl)-11-oxodibenzo[b,f][1,4]thiazepin-10(11H)-yl]acetamide.

Chembridge Corporation produces ethyl 1-(3-[(2,5-dimethyloxyphenyl)amino]-3-oxopropyl)-4-piperidinecarboxylate hydrochloride; ethyl 2-amino-4-methyl-5-({[2-(trifluoromethyl)phenyl]amino}carbonyl)-3-thiophenecarboxylate; N-[3-)1,3-benzodioxol-5-yl)-3-(3-methoxyphenyl)propyl]-N-benzylpropanamide; methyl {7-[(3-methoxybenzyl)oxy]-4,8-dimethyl-2-oxo-2H-chromen-3-yl}acetate;

Maybridge Chemical Company (A division of Thermo Fisher Scientific) produces ethyl 3-hydroxy-5-methyl-6-oxo-1-phenyl-1,6-dihydropyrano[2,3-c]pyrazole-4-carboxylate 1-(1,3-benzodioxol-5-ylmethyl)-4-[(4-chlorobenzyl)sulfonyl]piperazine; N′-(4-isopropylphenyl)-1-benzothiophene-2-carbohydrazide.

Sigma Aldrich produces 5-(4-bromophenyl)-3-hydroxy-4-(3-methyl-4-propoxybenzoyl)-1-[2-(4-morpholinyl)ethyl]-1,5-dihydro-2H-pyrrol-2-one; 5-(4-fluorophenyl)-3-hydroxy-4-(4-isobutoxy-3-methyl)-1-[2-(4-morpholinyl]-1,5-dihydro-2H-pyrrol-2one; and 5-(3-bromophenyl)-4-(3-fluoro-4-methoxybenzoyl)-3-hydroxy-1-[3-(4-morpholinyl)propyl]-1,5-dihydro-2H-pyrrol-2-one.

### Peptide Antagonists

The present disclosure also contemplates a peptide antagonist of Id1. Exemplary peptide antagonists are capable of forming a leucine zipper structure to thereby bind to Id1 and prevent Id1 binding to another protein, e.g., a HLH transcription factor. For example, the peptide does not bind to the other HLH transcription factor.

Sequences of exemplary peptide antagonists are set forth in any one of SEQ ID NOs: 5-16 or 28. For example, the antagonist comprises a sequence set forth in SEQ ID NO: 13. Additional exemplary peptide antagonists are described, for example, in Chen et al., J. Peptide Science, 16: 231-241, 2010.

The present disclosure also contemplates peptide antagonists comprising one or more non-naturally occurring amino acids or amino acid analogues. For example, a peptide antagonist may comprise one or more naturally occurring non-genetically encoded L-amino acids, synthetic L-amino acids or D-enantiomers of an amino acid. For example, the peptide comprises only D-amino acids. Exemplary non-coded amino acids include: hydroxyproline, β-alanine, 2,3-diaminopropionic acid, α-aminoisobutyric acid, N-methylglycine (sarcosine), ornithine, citrulline, t-butylalanine, t-butylglycine, N-methylisoleucine, phenylglycine, cyclohexylalanine, norleucine, naphthylalanine, pyridylananine 3-benzothienyl alanine 4-chlorophenylalanine, 2-fluorophenylalanine, 3-fluorophenylalanine, 4-fluorophenylalanine, penicillamine, 1,2,3,4-tetrahydro-tic isoquinoline-3-carboxylic acid β-2-thienylalanine, methionine sulfoxide, homoarginine, N-acetyl lysine, 2,4-diamino butyric acid, p-aminophenylalanine, N-methylvaline, homocysteine, homoserine, ε-amino hexanoic acid, δ-amino valeric acid, 2,3-diaminobutyric acid.

The present disclosure also encompasses retro-inverso peptide antagonists, e.g., in which two or more amino acids (e.g., all amino acids other than glycine) are D amino acids and the order of the D amino acids are reversed.

Peptide antagonists of the disclosure can be produced by recombinant means or synthetic means.

Recombinant Expression

Recombinant means generally comprise operably linking a nucleic acid encoding the peptide to a promoter to thereby form an expression construct, which can be an expression vector (e.g., a plasmid or phagemid). The present disclosure contemplates such an expression construct. The nucleic acid can be produced and/or isolated and cloned into an appropriate construct using methods known in the art and/or described in Ausubel et al (In: Current Protocols in Molecular Biology. Wiley Interscience, ISBN 047 150338, 1987) and/or (Sambrook et at (In: Molecular Cloning: Molecular Cloning: A Laboratory Manual, Cold Spring Harbor Laboratories, New York, Third Edition 2001).

Suitable promoters and/or expression vectors will be apparent to the skilled artisan based on the cell/expression system to be used. For example, typical promoters suitable for expression in a mammalian cell include, for example a promoter selected from the group consisting of, retroviral LTR elements, the SV40 early promoter, the SV40 late promoter, the CMV IE (cytomegalovirus immediate early) promoter, the EF_{1α} promoter (from human elongation factor 1α), the EM7 promoter, the UbC promoter (from human ubiquitin C). Examples of useful mammalian host cell lines include monkey kidney CV1 line transformed by SV40 (COS-7); human embryonic kidney line (293 or 293 cells); baby hamster kidney cells (BHK,); Chinese hamster ovary cells (CHO); African green monkey kidney cells (VERO-76); or myeloma cells (e.g., NS/0 cells). For example, the cells are CHO cells.

Other elements of expression constructs/vectors are known in the art and include, for example, enhancers, transcriptional terminators, polyadenylation sequences, nucleic acids encoding selectable or detectable markers and origins of replication.

Of course, the present disclosure contemplates expression in any cell, including bacterial cells, fungal cells, insect cells or plant cells.

Following production of a suitable expression construct, it is introduced into a suitable cell using any method known in the art. Exemplary methods include microinjection, transfection mediated by DEAE-dextran, transfection mediated by liposomes such as by using lipofectamine (Gibco, MD, USA) and/or cellfectin (Gibco, MD, USA), PEG-mediated DNA uptake, electroporation and microparticle bombardment such as by using DNA-coated tungsten or gold particles (Agracetus Inc., WI, USA) amongst others.

The present disclosure also encompasses recombinant cells expressing an antagonist of Id1.

The cells are then cultured under conditions known in the art to produce a peptide antagonist.

Cell free expression systems are also contemplated by the present disclosure, e.g., the TNT T7 and TNT T3 systems (Promega), the pEXP1-DEST and pEXP2-DEST vectors (Invitrogen).

Peptide Synthesis

A peptide of the disclosure can be synthesized using a chemical method known to the skilled artisan. For example, synthetic peptides are prepared using known techniques of solid phase, liquid phase, or peptide condensation, or any combination thereof, and can include natural and/or unnatural amino acids. Amino acids used for polypeptide synthesis may be standard Boc (Nα-amino protected Nα-t-butyloxycarbonyl) amino acid resin with the deprotecting, neutralization, coupling and wash protocols of the original solid phase procedure of Merrifield, J. Am. Chem. Soc., 85:2149-2154, 1963, or the base-labile Nα-amino protected 9-fluorenylmethoxycarbonyl (Fmoc) amino acids described by Carpino and Han, J. Org. Chem., 37:3403-3409, 1972.

Peptide Purification

Following production/expression/synthesis, a peptide is purified using a method known in the art. Such purification provides the peptide substantially free of conspecific protein, nucleic acids, lipids, carbohydrates, and the like. For example, the peptide will be in a preparation wherein more than about 90% (e.g. 95%, 98% or 99%) of the protein in the preparation is a peptide antagonist of Id1.

Standard methods of peptide purification are employed to obtain an isolated peptide, including but not limited to various high-pressure (or performance) liquid chromatography (HPLC) and non-HPLC peptide isolation protocols, such as size exclusion chromatography, ion exchange chromatography, phase separation methods, electrophoretic separations, precipitation methods, salting in/out methods, immunochromatography, and/or other methods.

### Antagonists of ID 1 Expression

### Small Molecule Antagonists

The present disclosure contemplates a small molecule antagonist of Id1 that reduces, prevents or inhibits expression of Id1. In one example, the antagonist is a cannabidiol compound comprising a structure set forth in Formula 6:

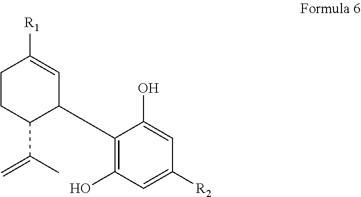

wherein
R₁ is an alkyl; and
R₂ is selected from a straight or branched alkyl having 5 to 12 carbon atoms; an —OR₃ group, wherein R₃ is a straight or branched alkyl having 5 to 9 carbon atoms or a straight or branched alkyl substituted at the terminal carbon atom by a phenyl group; or a —(CH₂)ₙ—O-alkyl group, wherein n is an integer from 1 to 7 and the alkyl group has 1 to 5 carbons.

In one preferred example, R₁ is CH₃ and R₂ is a straight alkyl having 5 carbon atoms (i.e. —O₅H₁₁).

A preferred cannabidiol comprises the structure set forth in Formula 7:

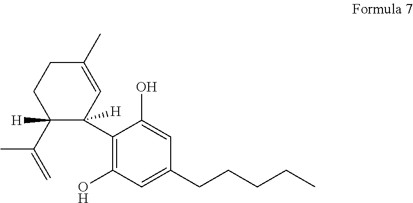 [SMILES: C=C(C)[C@@H]1CCC(C)=C[C@H]1c1c(O)cc(CCCCC)cc1O]

### Nucleic Acid Antagonists

In one example, an antagonist of Id1 expression binds to Id1 encoding nucleic acid and reduces, prevents or inhibits Id1 expression.

In one example, the antagonist is a nucleic acid-based antagonist. For example, the antagonist reduces, prevents or inhibits transcription and/or translation of an Id1 encoding nucleic acid, e.g., comprising a sequence set forth in SEQ ID NO: 1 and/or 3. In one example, the compound is an antisense polynucleotide, a ribozyme, a PNA, an interfering RNA, a siRNA, a microRNA

Antisense Polynucleotides

The term “antisense polynucleotide” shall be taken to mean a DNA or RNA, or combination thereof that is complementary to at least a portion of a mRNA encoding Id1 and capable of interfering with a post-transcriptional event such as mRNA translation. The use of antisense methods is known in the art.

An antisense polynucleotide of the disclosure will hybridize to a target polynucleotide under physiological conditions. Antisense polynucleotides include sequences that correspond to the structural genes or for sequences that effect control over gene expression or splicing. For example, the antisense polynucleotide may correspond to the targeted coding region of the genes of the disclosure, or the 5′-untranslated region (UTR) or the 3′-UTR or combination of these. It may be complementary in part to intron sequences, which may be spliced out during or after transcription, for example only to exon sequences of the target gene. The length of the antisense sequence should be at least 19 contiguous nucleotides, for example at least 50 nucleotides, and more for example at least 100, 200, 500 or 1000 nucleotides of a nucleic acid comprising a sequence set forth in SEQ ID NO: 1 or 3 or a structural gene encoding same. The full-length sequence complementary to the entire gene transcript may be used. The degree of identity of the antisense sequence to the targeted transcript should be at least 90%, for example 95-100%.

Exemplary antisense nucleotides comprise a sequence set forth in any one or more of SEQ ID NOs: 17-21. In one example of the sequence set forth in SEQ ID NO: 18, residues 1-5 and 19-23 are RNA and the remaining residues are DNA. Such an antisense polynucleotide is also known as a gapmer, in this case a 5-13-5 gapmer. For example, the RNA bases are 2′-O-methyl RNA bases and the DNA bases are phosphorothioate bases.

Additional exemplary antisense that reduce, prevent or inhibit Id1 expression are described in WO2009/089186 or U.S. Pat. No. 6,372,433.

In one example, the antisense polynucleotide is conjugated to a pancreatic targeting peptide, e.g., as described in WO2009/08916 and/or herein and/or a protein transduction domain.

Catalytic Polynucleotides

The term “catalytic polynucleotide/nucleic acid” refers to a DNA molecule or DNA-containing molecule (also known in the art as a “deoxyribozyme” or “DNAzyme”) or an RNA or RNA-containing molecule (also known as a “ribozyme” or “RNAzyme”) which specifically recognizes a distinct substrate and catalyses the chemical modification of this substrate. The nucleic acid bases in the catalytic nucleic acid can be bases A, C, G, T (and U for RNA).

Typically, the catalytic nucleic acid contains an antisense sequence for specific recognition of a target nucleic acid, and a nucleic acid cleaving enzymatic activity (also referred to herein as the “catalytic domain”). The types of ribozymes that are particularly useful in this disclosure are a hammerhead ribozyme and a hairpin ribozyme.

RNA Interference

RNA interference (RNAi) is useful for specifically inhibiting the production of a particular protein. This technology relies on the presence of dsRNAs that contain a sequence that is essentially identical to the mRNA of the gene of interest or part thereof, in this case an mRNA encoding an Id1 protein. Conveniently, the dsRNA can be produced from a single promoter in a recombinant vector or host cell, where the sense and anti-sense sequences are flanked by an unrelated sequence which enables the sense and anti-sense sequences to hybridize to form the dsRNA molecule with the unrelated sequence forming a loop structure. The design and production of suitable dsRNA molecules for the present disclosure is within the capacity of a person skilled in the art.

The length of the sense and antisense sequences that hybridize should each be at least 19 contiguous nucleotides, for example at least 30 or 50 nucleotides, such as at least 100, 200, 500 or 1000 nucleotides. The full-length sequence corresponding to the entire gene transcript may be used. In some examples, the lengths are 100-2000 nucleotides. The degree of identity of the sense and antisense sequences to the targeted transcript should be at least 85%, for example at least 90%, such as 95-100%.

Preferred small interfering RNA (“siRNA”) molecules comprise a nucleotide sequence that is identical to about 19-21 contiguous nucleotides of the target mRNA. For example, the siRNA sequence commences with the dinucleotide AA, comprises a GC-content of about 30-70% (for example, 30-60%, such as 40-60%, for example about 45%-55%), and does not have a high percentage identity to any nucleotide sequence other than the target in the genome of the mammal in which it is to be introduced, for example as determined by standard BLAST search.

### Pancreatic Targeting Compounds

In one example, an antagonist of Id1 activity and/or expression is linked to a compound that targets the pancreas.

By “linked” is meant two compositions of matter are either covalently or non-covalently bound to one another. For example, the two components are linked by a covalent bond.

By “pancreatic targeting” is meant that a compound preferentially binds to a component of a pancreas such that when administered to a subject the compound is localized to the pancreas to a higher level than other tissues. Relatively high levels of the compound may be found in tissues such as liver or kidney, however this is a result of clearance rather than localization mediated by the compound.

Preferred pancreatic targeting compounds are peptides. Exemplary peptides comprise a sequence set forth in any one of SEQ ID Nos: 11-25.

Additional exemplary pancreatic targeting peptides are described, for example, in US20090221505.

These peptides may also be modified to include modifications, e.g., as described herein.

In the case of pancreatic targeting peptides conjugated to nucleic acid Id1 antagonists, the peptide portions and the nucleic acid portions of the composition can be covalently coupled to one another via the heterobifunctional linker (HBL) that has reactivity with an amino and sulfhydryl groups. In certain examples, the heterobifunctional linker is a compound with a maleimide and a succinimide group. The nucleic acid is connected to the bifunctional linker using the maleimide activity of the linker and an amino functionality on the nucleic acid. The peptide is reacted with the bifunctional linker via succinimide portion and a sulfhydryl functionality on the peptide.

Methods for formation of peptide-oligonucleotide conjugates such as these are known. For example, the use of a small linker (4-(maleimidomethyl)-1-cyclohexane-carboxylic acid N-hydroxysuccinimide ether, SMCC) for this purpose is described in the Harrison et al., Nucleic Acids Res. 26: 3136-3145 (1998). Another method which can be used to conjugate nucleic acid and peptides uses 4-maleimidobutyric acid N-hydroxysuccinimide ester (GMBS) in place of SMCC. GMBS and SMCC have the same reactive groups, the maleimido group and the hydroxysuccinimide group, but differ in the intervening structure or spacer.

### Protein Transduction Domains

Some compounds must enter a cell to exert their biological activity. To facilitate peptide entry into a cell, the compound may be conjugated to (e.g., expressed as a fusion with o otherwise linked to) a protein transduction domain. As used herein, the term “protein transduction domain” shall be taken to mean a peptide or protein that is capable of enhancing, increasing or assisting penetration or uptake of a compound conjugated to the protein transduction domain into a cell either in vitro or in vivo. Those skilled in the art will be aware that synthetic or recombinant peptides can be delivered into cells through association with a protein transduction domain such as the TAT sequence from HIV (SEQ ID NO: 26) or the Penetratin sequence from the Antennapaedia homeodomain protein (SEQ ID NO: 27) (see, for example, Temsamani and Vidal, Drug Discovery Today 9: 1012-1019, 2004, for review).

Additional suitable protein transduction domains are described, for example, in US20040197867.

### Screening Assays

Antagonists of the disclosure are readily screened for biological activity.

An exemplary in vitro method for determining the effect of the antagonist is to contact it to β-cells (e.g., a β-cell line such as MIN6 or HC-9) with the antagonist and assessing its effect, e.g., on insulin secretion, such as in response to glucose stimulation. Exemplary assays for measuring insulin secretion are known in the art and include, for example commercially available enzyme-linked immunosorbent assays (ELISAs) as exemplified herein. An antagonist that increases insulin secretion in response to glucose is considered a therapeutic/prophylactic compound.

Alternatively, or in addition, an assay detects β cell line proliferation in response to different concentrations of glucose in the presence or absence of an antagonist. Methods for assessing cell proliferation are known in the art and include, for example, ¹³H thymidine incorporation, BrdU incorporation or a MTT assay.

Alternatively, or in addition, antagonist is administered to an accepted animal model of an abnormality of glucose metabolism, e.g., type 2 diabetes. For example, the present inventors have used the high fat fed murine model of type 2 diabetes. Other models of type 2 diabetes include, for example, high fat fed streptozotocin-treated rodents (Mu et al., Diabetes, 55: 1695-1704, 2006), db/db mice (commercially available), animals transgenic for islet amyloid polypeptide (e.g., as reviewed in Matveyenko et al., ILAR J. 47: 225-33, 2006) or other model as known in the art. A symptom of type 2 diabetes is then assessed, e.g., using a glucose tolerance test or GSIS assessment using islets isolated from the model to assess the effect of the antagonist.

### Pharmaceutical Compositions

The antagonist of the present disclosure (syn. active ingredient) is useful for parenteral, topical, oral, or local administration, aerosol administration, or transdermal administration, for prophylactic or for therapeutic treatment.

Formulation of an antagonist to be administered will vary according to the compound, route of administration and formulation (e.g., solution, emulsion, capsule) selected. An appropriate pharmaceutical composition comprising an antagonist to be administered can be prepared in a physiologically acceptable carrier. A mixture of antagonists can also be used. For solutions or emulsions, suitable carriers include, for example, aqueous or alcoholic/aqueous solutions, emulsions or suspensions, including saline and buffered media. Parenteral vehicles can include sodium chloride solution, Ringer's dextrose, dextrose and sodium chloride, lactated Ringer's or fixed oils. A variety of appropriate aqueous carriers are known to the skilled artisan, including water, buffered water, buffered saline, polyols (e.g., glycerol, propylene glycol, liquid polyethylene glycol), dextrose solution and glycine. Intravenous vehicles can include various additives, preservatives, or fluid, nutrient or electrolyte replenishers (See, generally, Remington's Pharmaceutical Science, 16th Edition, Mack, Ed. 1980). The compositions can optionally contain pharmaceutically acceptable auxiliary substances as required to approximate physiological conditions such as pH adjusting and buffering agents and toxicity adjusting agents, for example, sodium acetate, sodium chloride, potassium chloride, calcium chloride and sodium lactate. The antagonist of this disclosure can be lyophilized for storage and reconstituted in a suitable carrier prior to use according to art-known lyophilization and reconstitution techniques.

The optimum concentration of the active ingredient(s) in the chosen medium can be determined empirically, according to procedures known to the skilled artisan, and will depend on the ultimate pharmaceutical formulation desired.

The dosage ranges for the administration of the antagonist of the disclosure are those large enough to produce the desired effect. For example, the composition comprises a therapeutically or prophylactically effective amount of the antagonist.

As used herein, the term “effective amount” shall be taken to mean a sufficient quantity of the antagonist to inhibit/reduce/prevent expression and/or activity of Id1 in a subject. The skilled artisan will be aware that such an amount will vary depending on, for example, the antagonist and/or the particular subject and/or the type or severity of a condition being treated. Accordingly, this term is not to be construed to limit the disclosure to a specific quantity, e.g., weight or number of antagonists, rather the present disclosure encompasses any amount of the antagonist that is sufficient to achieve the stated purpose.

As used herein, the term “therapeutically effective amount” shall be taken to mean a sufficient quantity of the antagonist to reduce or inhibit one or more symptoms of an abnormality of glucose metabolism.

As used herein, the term “prophylactically effective amount” shall be taken to mean a sufficient quantity of the antagonist to prevent or inhibit or delay the onset of one or more detectable symptoms of an abnormality of glucose metabolism.

The dosage should not be so large as to cause adverse side effects, such as hyper viscosity syndromes, pulmonary edema, congestive heart failure, and the like. Generally, the dosage will vary with the age, condition, sex and extent of the disease in the patient and can be determined by one of skill in the art. The dosage can be adjusted by the individual physician in the event of any complication. Dosage can vary from about 0.1 mg/kg to about 300 mg/kg, such as from about 0.2 mg/kg to about 200 mg/kg, for example from about 0.5 mg/kg to about 20 mg/kg, in one or more dose administrations daily, for one or several days.

One or more antagonists of the present disclosure can be administered to an individual by an appropriate route, either alone or in combination with (before, simultaneous with, or after) another drug or agent. For example, the antagonist can be administered together with insulin and/or a GLP-1 analog and/or a DPPIV antagonist and/or a stem cell and/or an anti-inflammatory and/or a painkiller. The antagonist of the present disclosure can be used as separately administered compositions given in conjunction with antibiotics and/or antimicrobial agents.

It will be appreciated by those skilled in the art that peptide antagonists or some nucleic acid antagonists (e.g., siRNA or shRNA or microRNA) of the present disclosure may be introduced into a subject by administering an expression construct of the disclosure or a cell expressing the antagonist. A variety of methods can be used for introducing a nucleic acid encoding the antagonist into a target cell in vivo. For example, the naked nucleic acid may be injected at the target site, may be encapsulated into liposomes, or may be introduced by way of a viral vector.

Similarly, a cell expressing and secreting a peptide can be administered to a subject.

### Diagnostic Assays

The level of expression of Id1 can also be assessed at the protein or nucleic acid level for diagnose an abnormality of glucose metabolism (e.g., type 2 diabetes).

### Protein-Based Assays

Ligands for Diagnosis

As used herein the term “ligand” shall be taken in its broadest context to include any chemical compound, polynucleotide, peptide, protein, lipid, carbohydrate, small molecule, natural product, polymer, etc. that is capable of selectively binding, whether covalently or not, a marker described herein. The ligand may bind to its target via any means including hydrophobic interactions, hydrogen bonding, electrostatic interactions, van der Waals interactions, pi stacking, covalent bonding, or magnetic interactions amongst others. This term includes antibodies and fragments thereof.

Antibodies against Id1 are commercially available from, e.g., Abcam, Abgent or Abnova. Alternatively, a suitable antibody is produced using a method known in the art.

Assay Formats

As will be apparent to the skilled person from the foregoing, an immunoassay is a preferred assay format for diagnosing an abnormality of glucose metabolism in a subject. The present disclosure contemplates any form of immunoassay, including Western blotting, enzyme-linked immunosorbent assay (ELISA), fluorescence-linked immunosorbent assay (FLISA), competition assay, radioimmunoassay, lateral flow immunoassay, flow-through immunoassay, electrochemiluminescent assay, nephelometric-based assays, turbidometric-based assay, and fluorescence activated cell sorting (FACS)-based assays.

One form of a suitable immunoassay is, for example, an ELISA or FLISA.

In one form such an assay involves immobilizing an antibody or ligand that binds to Id1 onto a solid matrix, such as, for example a polystyrene or polycarbonate microwell or dipstick, a membrane, or a glass support (e.g. a glass slide). A test sample is then brought into direct contact with the antibody or ligand and any antigen in the sample is bound or captured. Following washing to remove any unbound protein in the sample, an antibody or ligand that binds to another epitope of Id1 is brought into direct contact with the captured protein. This antibody/ligand is generally labeled with a detectable reporter molecule, such as for example, an enzyme (e.g. horseradish peroxidase (HRP)), alkaline phosphatase (AP) or β-galactosidase. Alternatively, a second labeled antibody/ligand can be used that binds to the first antibody. Following washing to remove any unbound antibody the detectable marker is detected by the addition of a substrate, such as for example hydrogen peroxide, TMB, or toluidine, or 5-bromo-4-chloro-3-indol-b eta-D-galaotopyrano side (x-gal).

The level of the antigen in the sample is then determined using a standard curve that has been produced using known quantities of the marker or by comparison to a control sample.

In the case of FLISA, a fluorescent label is used to determine the level of a labeled ligand or antibody in a sample. A FLISA is performed essentially as described supra for the ELISA assay, however, a substrate is not required to detect the bound labeled ligand or antibody. Rather, following washing to remove any unbound ligand/antibody the sample is exposed to a light source of the appropriate wavelength and the level of fluorescence emitted by each sample determined.

In the above, ELISA or FLISA either of the antibodies/ligands can be substituted with a protein of the disclosure.

The assays described above are readily modified to use chemiluminescence or electrochemiluminescence as the basis for detection.

As will be apparent to the skilled artisan, other detection methods based on an immunosorbent assay are useful in the performance of the present disclosure. For example, an immunosorbent method based on the description supra using a radiolabel for detection, or a gold label (e.g. colloidal gold) for detection, or a liposome, for example, encapsulating NAD+ for detection (e.g., as described in Kumada et al., Journal of Chemical Engineering of Japan, 34: 943-947, 2001) or an acridinium linked immunosorbent assay.

As will be apparent to the skilled artisan from the preceding description, in one example, the method of the disclosure comprises contacting a sample from the subject with an antibody/ligand that binds to Id1 such that a complex forms and detecting the complex.

In one example, the method of the disclosure comprises:

(i) contacting a sample from the subject with a first antibody/ligand that binds to Id1 such that a complex forms; and
(ii) contacting the complex at (i) with a second antibody/ligand that binds to Id1 such that a complex forms; and
(iii) detecting the complex formed at (ii).

In one example, either of the ligands is or was previously immobilized on a solid support to facilitate capture or binding of the antigen in a body fluid.

In another example, the antibody or ligand that is not immobilized on a solid support is or was previously labeled with a detectable marker or label to facilitate determining the level of the second bound antibody or ligand.

In some examples of the disclosure, the level of Id1 is determined using a surface plasmon resonance detector (e.g., BIAcore™, Pharmacia Biosensor, Piscataway, N.J.), a flow through device, for example, as described in U.S. Pat. No. 7,205,159; a micro- or nano-immunoassay device (e.g., as described in US20030124619); a lateral flow devices (e.g., as described in US20040228761 or US20040265926); a fluorescence polarization immunoassay (FPIA e.g., as described in U.S. Pat. No. 4,593,089 or 4,751,190); or an immunoturbidimetric assay (e.g., as described in U.S. Pat. No. 5,571,728 or 6,248,597).

### Nucleic Acid-Based Detection Assays

In another example, the level of an Id1 nucleic acid is detected. Exemplary assays for such detection include quantitative RT-PCR, NASBA, TMA or ligase-chain reaction.

Methods of RT-PCR are known in the art and described, for example, in Dieffenbach and Dveksler (Eds) (In: PCR Primer: A Laboratory Manual, Cold Spring Harbour Laboratories, NY, 1995).

Methods of TMA or self-sustained sequence replication (3SR) use two or more oligonucleotides that flank a target sequence, a RNA polymerase, RNase H and a reverse transcriptase. One oligonucleotide (that also comprises a RNA polymerase binding site) hybridizes to an RNA molecule that comprises the target sequence and the reverse transcriptase produces cDNA copy of this region. RNase H is used to digest the RNA in the RNA-DNA complex, and the second oligonucleotide used to produce a copy of the cDNA. The RNA polymerase is then used to produce a RNA copy of the cDNA, and the process repeated.

NASBA systems relies on the simultaneous activity of three enzymes (a reverse transcriptase, RNase H and RNA polymerase) to selectively amplify target mRNA sequences. The mRNA template is transcribed to cDNA by reverse transcription using an oligonucleotide that hybridizes to the target sequence and comprises a RNA polymerase binding site at its 5′ end. The template RNA is digested with RNase H and double stranded DNA is synthesized. The RNA polymerase then produces multiple RNA copies of the cDNA and the process is repeated.

Clearly, the hybridization to and/or amplification of a nucleic acid using any of these methods is detectable using, for example, electrophoresis and/or mass spectrometry. In this regard, one or more of the probes/primers and/or one or more of the nucleotides used in an amplification reactions may be labeled with a detectable marker to facilitate rapid detection of a marker, for example, a fluorescent label (e.g. Cy5 or Cy3) or a radioisotope (e.g. ³²P). Alternatively, amplification of a nucleic acid may be continuously monitored using a melting curve analysis method, such as that described in, for example, U.S. Pat. No. 6,174,670.

### Samples and Control Samples

As will be apparent to the skilled artisan, some of the examples described herein require some degree of quantification to determine the level of Id1. Such quantification may be determined by the inclusion of a suitable control sample in an assay of the disclosure.

In one example, a suitable control sample is a sample that is derived from a healthy subject or a normal subject.

In the present context, the term “healthy subject” shall be taken to mean an individual who is known not to suffer from an abnormality of glucose metabolism, e.g., type 2 diabetes.

The term “normal subject” shall be taken to mean an individual having a normal level of Id1 in a sample compared to a population of individuals.

The present disclosure also contemplates the control sample as being a data set obtained from a normal and/or healthy subject or a population of normal and/or healthy subjects.

In one example, a method of the disclosure additionally comprises determining the level of Id1 in a control sample, e.g., using a method described herein.

In one example, a sample from the subject and a control sample are assayed at approximately or substantially the same time.

In one example, the sample from the subject and the control sample are assayed using the same method of the disclosure as described herein in any one or more examples to allow for comparison of results.

### Kits

The present disclosure additionally comprises a kit comprising one or more of the following:

(i) an antagonist of Id1 or nucleic acid encoding same;
(ii) a cell of the disclosure;
(iii) a pharmaceutical composition of the disclosure; and/or
(iv) a ligand or antibody that binds to Id1.

In the case of a kit for detecting Id1, the kit can additionally comprise a detection means, e.g., linked to a ligand or protein of the disclosure.

In the case of a kit for therapeutic/prophylactic use, the kit can additionally comprise a pharmaceutically acceptable carrier or diluent.

Optionally a kit of the disclosure is packaged with instructions for use in a method described herein according to any example.

The present disclosure includes the following non-limiting Examples.

### Example 1

### Materials and Methods

### 1.1 Human Pancreas Staining of Id1

Formalin-fixed, paraffin-embedded human pancreata were used to construct tissue microarrays consisting of 2-mm diameter tissue core biopsies containing islets. Serial sections (4 μm) were dewaxed in xylene and rehydrated in a series of graded alcohols. To unmask antigens slides were boiled in Tris-EDTA (pH 8) for 15 min. Slides were stained for Id1 (C-20, sc-488, dilution 1:500; Santa Cruz Biotechnology, Santa Cruz, Calif., USA) and insulin (12018; dilution 1:200; Sigma; St. Louis, Mo., USA) overnight at 4° C. The primary antibody was visualised using Alexa Fluor 488 and 555 Dyes (Invitrogen).

### 1.2 Mice

Wildtype (C57BL/6/129/Sv), Id1^{-/-} and Id3^{-/-} mice (Lyden et al., Nature, 401: 670-677, 1999) were kept under conventional conditions with free access to food and water. Mice were fed ad libitum with either a standard chow diet (8% calories from fat, 2.6 kcal/g, Gordon's Speciality Stockfeeds, Yanderra, Australia) or a high-fat diet containing lard/sucrose (45% calories from fat, 4.7 kcal/g, based on Rodent diet D12451; Research Diets, New Brunswick, N.J.).

Food intake and body weight were measured for the determination of energy intake (expressed as kcal/g bodyweight).

Blood collected in EDTA via a terminal heart bleed was used for measurement of blood glucose and plasma insulin, glucagon, triglyceride and non-esterified fatty acid (NEFA) levels. An insulin resistance index, homeostasis model assessment of insulin resistance (HOMA-IR), was calculated from glucose and insulin levels [glucose concentration (mM)×insulin concentration (mU/1)÷22.5].

### 1.3 Glucose and Insulin Tolerance Tests

Intraperitoneal glucose tolerance tests (i.p. GTT, 2 g/kg glucose, Phebra, Lane cove, Australia) and insulin tolerance tests (i.p. ITT, 0.75 units/kg insulin, Actrapid Penfill, Novo Nordisk A/S, Baulkham Hills, Australia) were performed in conscious male mice after 6 h of fasting. Blood samples were taken via tail prick at 0, 15, 30, 45, 60 and 90 min for assessment of glucose concentrations (i.p. GTT and ITT) and at 0, 15, 30 and 45 min for assessment of insulin concentrations (i.p. GTT). Glucose was measured using an Accu-Chek Performa glucose monitor (Roche Diagnostics, Castle Hill, Australia). Insulin was measured using an ELISA (Crystal Chem. Inc., Downers Grove, Ill.) with blood collected using 5 μl Accu-Cap heparinized capillaries (Bilbate, Daventry, Northamptonshire, UK). Plasma glucagon levels were measured using a radioimmunoassay (Millipore, Billerica, Mass.). Plasma triglyceride levels were measured using an enzymatic colorimetric method (GPO-PAP reagent, Roche Diagnostics) with glycerol as standard. Plasma NEFA levels were measured by an acyl-CoA oxidase-based colorimetric method (Wako Pure Chemical Industries, Osaka, Japan).

### 1.4 Measurement of β-Cell Mass, Islet Number and Apoptosis

Pancreata were removed, fixed in paraformaldehyde and embedded in paraffin. Sections (5 μM thick) were stained for insulin (12018; dilution 1:200; Sigma Aldrich) and counterstained with hematoxylin. Whole slide digital images were captured using Aperio Scansope XT (Aperio Technologies, Vista, Calif.). β-cell mass and islet number were quantified using ImageScope software (Aperio Technologies). Four sections separated by at least 100 μm were used for each mouse. β-cell mass was calculated from relative cross-sectional β-cell area and total pancreas mass. Islet number was quantified as number of islets per mm2 of total pancreas. Apoptosis was assessed in pancreas sections using the terminal deoxynucleotidyl transferase-mediated dUTP nick end-labeling technique (In Situ Cell Death Detection Kit, POD, Roche).

### 1.5 Mouse Islet Isolation and Insulin Secretion Assay

Islets were isolated by in situ pancreas perfusion with a solution containing liberase RI (Roche Diagnostics), followed by incubation at 37° C. in a water bath and further separation using a Ficoll-Paque PLUS® gradient (GE Healthcare Bio-Sciences, Uppsala, Sweden) and handpicking under a stereomicroscope. Insulin secretion assay was performed immediately after islet isolation. Islets were washed in Krebs-Ringer HEPES buffer (KRHB; containing 5 mM NaHCO3, 1 mM CaCl2, 2.8 mM glucose, 10 mM HEPES, and 10% FCS). Groups of five islets, with at least 4 replicates per animal, were pre-incubated for 30 min at 37° C. in KRHB and then incubated for 1 h in KRHB containing 2.8 or 16.7 mM. Insulin was measured in an aliquot of the buffer by radioimmunoassay (Millipore, Billerica, Mass.).

### 1.6 Cell Culture and Transfection

To test the effects of Id1 overexpression, mouse Id1 cDNA was cloned into the expression vector pcDNA-DEST40 (Invitrogen). Id1-DEST40 or control pmaxGFP (green fluorescent protein) were transfected into MIN6 cells by nucleofector (Amaxa Biosystems, Cologne, Germany). Cells were seeded at 5×10⁵ cells in 1 ml of DMEM per well in a 12-well plate. Two days after transfection, cells were washed in KRB buffer containing 2.8 mM glucose, and then preincubated for a further 30 min in 1 ml of the same medium at 37° C. This buffer was then replaced with 1 ml of prewarmed KRB containing 2.8 or 16.7 mM glucose, and incubated for a further 60 min at 37° C. An aliquot was then removed for analysis of insulin content by radioimmunoassay. The cell monolayers were washed in PBS and then extracted for measurement of total insulin content.

For Id1 silencing experiments, MIN6 cells were passaged in 150 cm² flasks with 25 ml DMEM (Invitrogen, Carlsbad, Calif., USA) containing 25 mM glucose, 10 mM HEPES, 10% FCS, 50 U/ml penicillin and 50 μg/ml streptomycin. Cells were seeded at 2×10⁵ in 24-well plates. Id1 ON-TARGETplus SMARTpool siRNA or negative control nontargeting siRNA were transfected into MIN6 cells using DharmaFECT Transfection Reagent 3 (Dharmacon, Lafayette, Colo.). Following 24 hours culture, cells were treated with either 0.92% BSA or 0.92% BSA coupled to 0.4 mM palmitate for 48 h substantially as previously described (Busch Diabetes, 51: 977-987, 2002). For insulin secretion assays, cells were pre-incubated for 30 min at 37° C. in KRHB and then incubated for 1 h in KRHB containing 2.8 or 25 mM glucose. 0.4 mM palmitate was present during the 1 h incubation. Insulin was measured in an aliquot of the buffer by radioimmunoassay (Millipore, Billerica, Mass.).

### 1.7 RNA Anaylsis

Total RNA was extracted from MIN6 cells or islets using RNeasy Mini Kit (Qiagen, Doncaster, Australia) and cDNA was synthesised using QuantiTect Reverse Transcription Kit (Qiagen, Victoria, Australia). Real-time PCR was performed using SYTO 9 green-fluorescent nucleic acid stain (Invitrogen, Mulgrave, Australia), SensiMix dT (Bioline, Alexandria, Australia) and oligonucleotide primers (sequences listed in Table 1) in a LightCycler (Roche Diagnostics, Castle Hill, Australia). The value obtained for each specific product was normalized to the control gene (cyclophilin A) and expressed as a percent of the value in control extracts.

TABLE 1
Primers used for mRNA expression analysis

| mRNA | 5′ Oligonucleotide | SEQ ID NO | 3′ Oligonucleotide | SEQ ID NO |
|---|---|---|---|---|
| Cyclophilin A (Ppia) | TGTGCCAGGGTGGTGACTTTAC | 29 | TGGGAACCGTTTGTGTTTGG | 30 |
| Id1 | TTGGTCTGTCGGAGCAAAGC | 31 | GCAGGTCCCTGATGTAGTCGATTAC | 32 |
| Insulin | TCTTCTACACACCCATGTCCC | 33 | GGTGCAGCACTGATCTAC | 34 |
| Glucagon | ATGAATGAAGACAAACGCCAC | 35 | ACTTCTTCTGGGAAGTCTCGC | 36 |
| Pdx1 | CGGACATCTCCCCATACG | 37 | AAAGGGAGCTGGACGCGG | 38 |
| Beta2 (Neurod1) | ACTCCAAGACCCAGAAACTGTC | 39 | ACTGGTAGGAGTAGGGATGCAC | 40 |
| Glut2 (Slc2a2) | CATTCTTTGGTGGGTGGC | 41 | CCTGAGTGTGTTTGGAGCG | 42 |
| Pc (Pcx) | GTTCCGTGTCCGAGGTGTAAAG | 43 | CGCAGAAGGATGTCCCTGAAAC | 44 |
| Gk (Gck) | CATTGAATCAGAGGAGGGCAGC | 45 | TAGTGGACTGGGAGCATTTGTGGG | 46 |
| Gpr40 (Ffar1) | TATTCCTGGGGTGTGTGTGTGG | 47 | CCAAGGGCAGAAAGAAGAGCAG | 48 |
| BiP (Hspa5) | AGGACAAGAAGGAGGATGTGGG | 49 | ACCGAAGGGTCATTCCAAGTG | 50 |
| Chop (Ddit3) 176 | TTCACTACTCTTGACCCTGCGTC | 51 | CACTGACCACTCTGTTTCCGTTTC | 52 |
| Ho-1 (Hmox1) | CCACACAGCACTATGTAAAGCGTC | 53 | GTTCGGGAAGGTAAAAAAAGCC | 54 |

### 1.8 Statistical Analysis

All results are presented as means±SEM. Statistical analyses were performed using Student's t test or ANOVA with Bonferroni post hoc tests.

### Example 2

### Id1 Expression is Increased in Islets of Human Subjects Suffering from Type 2 Diabetes

A human pancreas tissue microarray was constructed using formalin fixed, paraffin-embedded pancreas from non-diabetic and type 2 diabetic patients. In sections of the tissue array, islets from 7 of 7 non-diabetic subjects showed only occasional Id1 staining, with Id1 expression absent from most cells within the islets. In contrast, in islets from 6 of 7 T2D subjects, the majority of islet cells displayed Id1 staining. These data indicate that Id1 expression is up-regulated in islets of patients suffering from type 2 diabetes.

### Example 3

### Metabolic Characteristics of Wild-Type and Id1^{-/-} Mice Fed a Chow or a High-Fat Diet

In mice fed a chow diet, body weight, epididymal fat pad weight, liver weight and energy intake were not significantly different in wild-type and Id1^{-/-} mice. Similarly, blood glucose and plasma insulin, glucagon, triglyceride and NEFA levels were unchanged, although a trend towards slightly lower blood glucose levels was observed in Id1^{-/-} mice. Thus, Id1^{-/-} mice appear to develop without obvious metabolic abnormalities. High-fat feeding of wild-type and Id1^{-/-} mice for 6 weeks led to significant increases in body weight, fat pad weight and energy intake, which was similar in both genotypes. Liver weight was not affected by high-fat feeding in either genotype. Plasma insulin and triglyceride levels were significantly increased by high-fat feeding in both genotypes, whereas plasma glucagon and NEFA levels were unchanged. There was a tendency for slightly higher blood glucose levels in both wild-type and Id1^{-/-} mice after high-fat feeding. HOMA-IR scores (calculated from blood glucose and plasma insulin levels) were increased by fat feeding irrespective of genotype.

### Example 4

### Id1^{-/-} Mice Exhibit Improved Glucose Tolerance

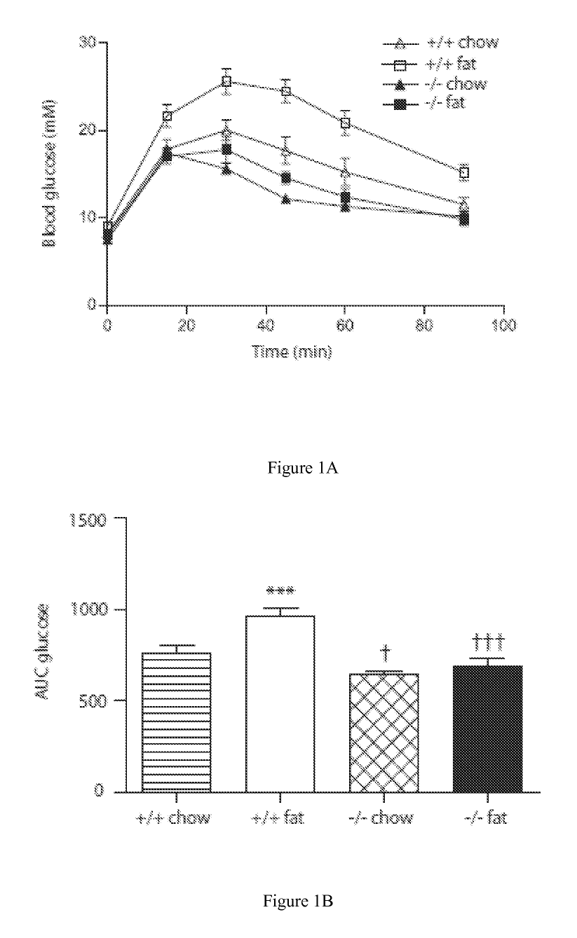

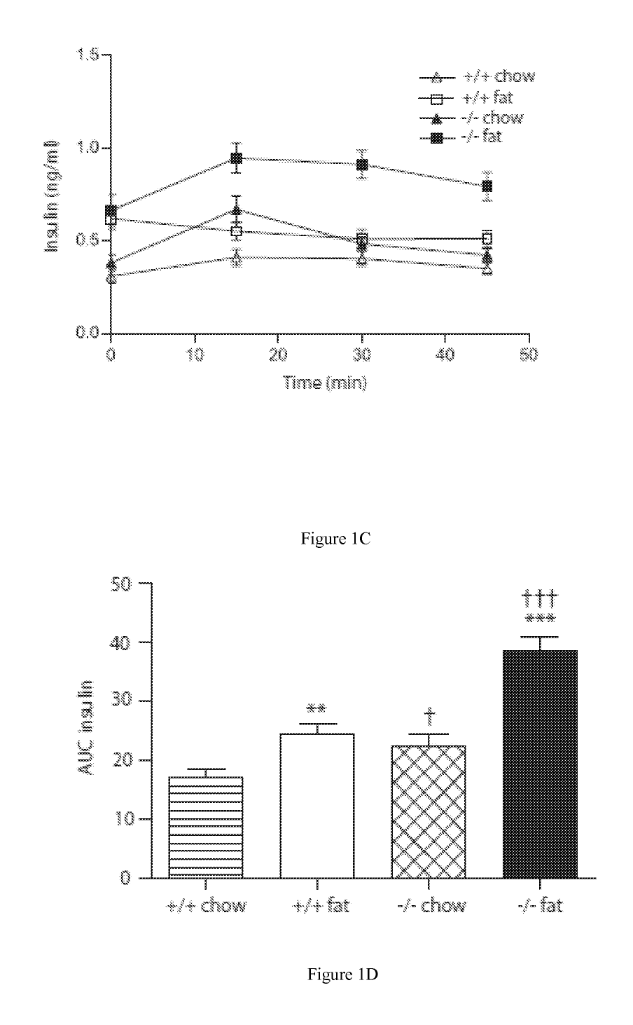

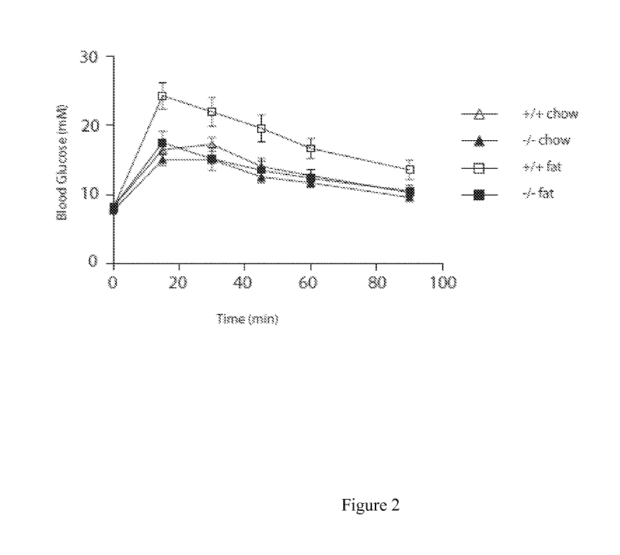

To determine whether or not Id1 plays a role in the regulation of glucose tolerance, i.p. GTT were performed in wild-type and Id1^{-/-} mice fed a chow or a high-fat diet for 6 weeks. After 6 h of fasting, blood glucose levels were similar among the diets and genotypes (indicated at 0 min, FIG. 1A). After i.p. glucose administration in mice fed a chow diet, blood glucose levels (FIG. 1A) and the resultant area under the curve (AUC) for glucose values from 0-90 min (FIG. 1B) were significantly reduced in Id1^{-/-} mice compared to wild-type mice. These data indicate that Id1 inhibition leads to improved glucose tolerance in chow-fed mice (FIGS. 1A and B). Compared to chow-fed mice, high-fat feeding of wild-type mice led to significantly increased blood glucose levels following the i.p. bolus (FIG. 1A, B), indicating that a 6-week exposure to a high-fat diet results in marked glucose intolerance. Id1^{-/-} mice were considerably protected from high-fat diet-induced glucose intolerance (FIGS. 1A and B). Following the glucose challenge, the blood glucose levels of fat-fed Id1^{-/-} mice were only slightly elevated compared to levels in chow-fed Id1^{-/-} mice, and they remained below the range observed in chow-fed wild-type mice (FIGS. 1A and 1). These data indicate that inhibition of Id1 confers protection against diet-induced glucose intolerance. This effect in Id1^{-/-} mice was also observed after a more prolonged 18-week period of high-fat feeding (FIG. 2).

### Example 5

### Improved Glucose Tolerance in Id1^{-/-} Mice is Associated with Increased Insulin Levels

During the i.p. GTT assays, insulin levels (FIG. 1C) and the resultant area under the curve (AUC) for insulin (FIG. 1D) were significantly increased in Id1^{-/-} mice compared to wild-type controls. This was particularly evident after high-fat feeding. Compared to chow-fed controls, fat-fed wild-type mice exhibited higher fasting insulin levels (indicated at 0 min, FIG. 1C), but these did not increase further following i.p. glucose administration (FIG. 1C), despite the presence of marked hyperglycemia (FIG. 1A). In contrast, in fat-fed Id1^{-/-} mice insulin levels were significantly increased following i.p. glucose administration (FIGS. 1C and D). Without being bound by any theory or mode of action, these data suggest that the ability of Id1^{-/-} mice to improve glucose tolerance may be associated with enhanced circulating insulin levels, especially following high-fat diet-induced insulin resistance.

### Example 6

### Glucose Tolerance is Unaltered in Id3^{-/-} Mice

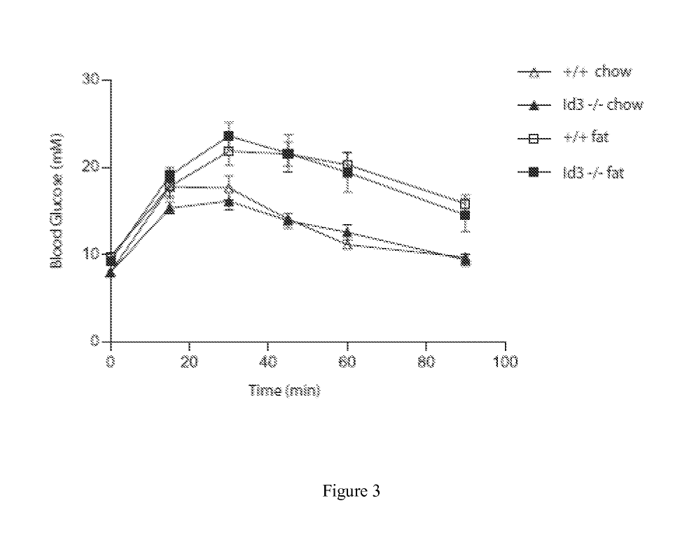

Id3 is closely related to Id1. To determine whether Id3 plays a role in the regulation of glucose tolerance, i.p. GTT were performed in wild-type and Id3^{-/-} mice fed a chow or a high-fat diet for 6 weeks. Blood glucose levels during the i.p. GTT were similar in wild-type and Id3^{-/-} mice fed a chow diet (FIG. 3).

Furthermore, after 6 weeks of high-fat feeding, blood glucose levels during the i.p. GTT were increased in both genotypes compared to corresponding values in chow-fed mice (FIG. 3), suggesting that the deletion of Id3 does not affect diet-induced glucose intolerance. Taken together, the data presented in Examples 5 and 6 indicate a specific role of Id1, and not the closely related Id3 family member, in the regulation of glucose tolerance. This is not to say that an inhibitor of Id1 and Id3 will not treat a glucose metabolism disorder, merely that Id1 inhibition provides a benefit.

### Example 7

### Effect of Id1 Deletion on Insulin Action

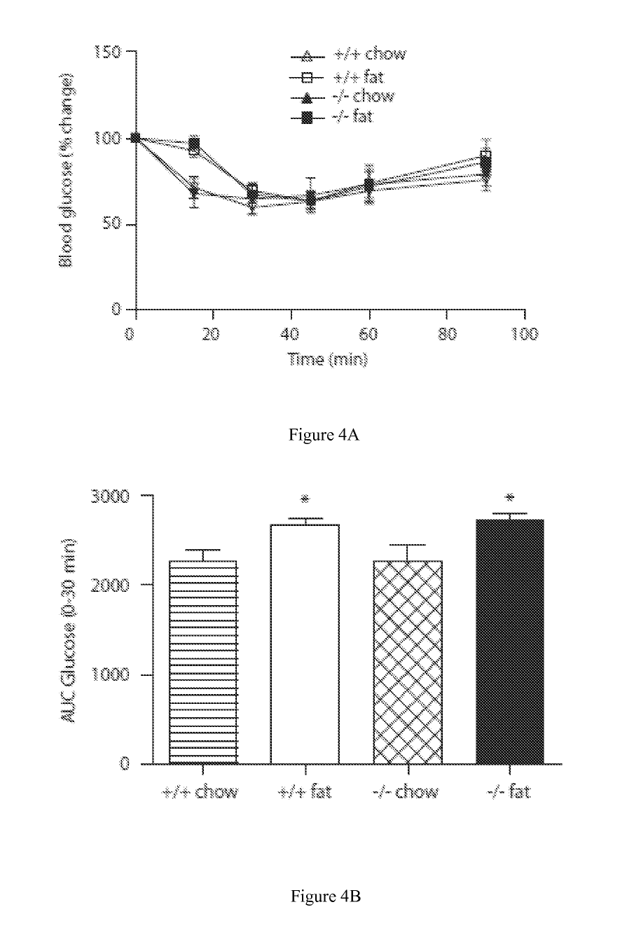

To investigate whether or not changes in insulin action contribute to the improved glucose tolerance in Id1^{-/-} mice, i.p. ITT were performed in wild-type and Id1^{-/-} mice fed a chow or a high-fat diet. Chow-fed wild-type and Id1^{-/-} mice exhibited similar time course changes in blood glucose levels after insulin injection (FIG. 4A). Accordingly, the AUC of glucose values from 0-30 min following insulin injection were similar in both genotypes (FIG. 4B). After high-fat feeding, the blood glucose response to insulin was delayed in both wild-type and Id1^{-/-} mice, indicating that diet-induced insulin resistance was not affected by deletion of Id1. Without being bound by any theory or mode of action, these data suggest that the improved glucose tolerance in Id1^{-/-} mice may be a consequence of increased insulin levels, rather than changes in insulin action.

### Example 8

### Effect of Id1 Deletion on β-Cell Mass or Islet Number

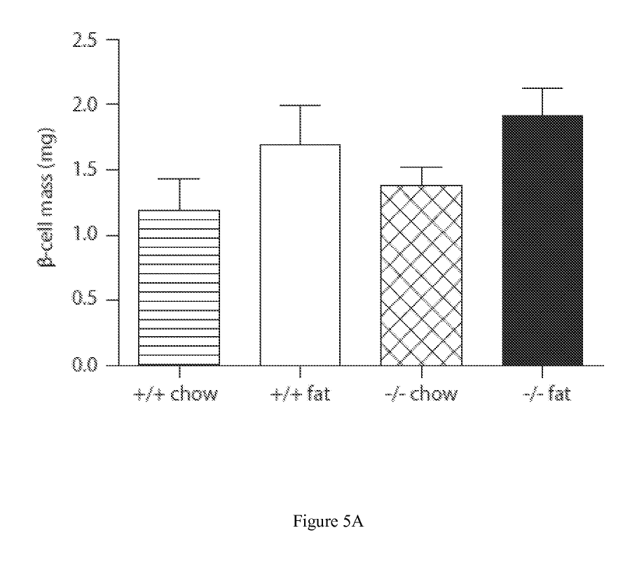

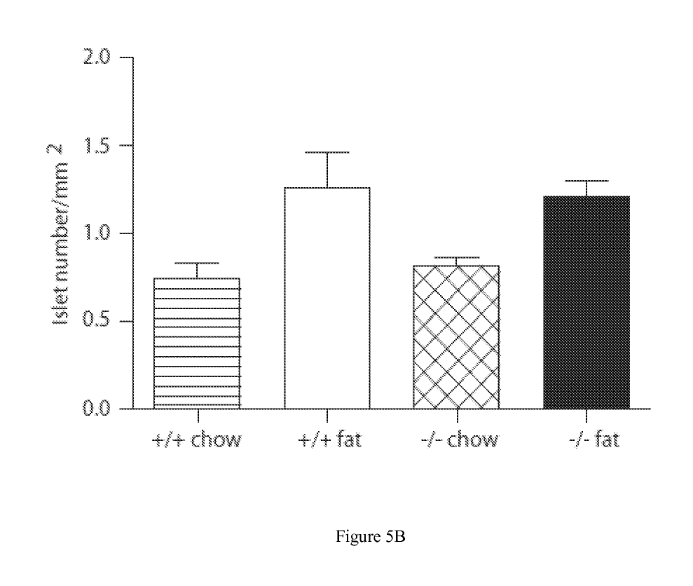

To determine whether or not Id1 plays a role in the regulation of β-cell mass, morphometric analyses of pancreas sections from wild-type and Id1^{-/-} mice fed a chow or a high-fat diet were performed. There were no differences in β-cell mass or in the number of islets between wildtype and Id1^{-/-} mice fed a chow diet (FIGS. 5A and B). The level of β-cell apoptosis was also examined, but no differences were detected between the genotypes on both diets. Without being bound by any theory or mode of action, these data suggest that changes in β-cell capacity may not contribute to the increased insulin levels in Id1^{-/-} mice.

### Example 9

### Islets from Id1^{-/-} Mice Display Enhanced Insulin Secretion

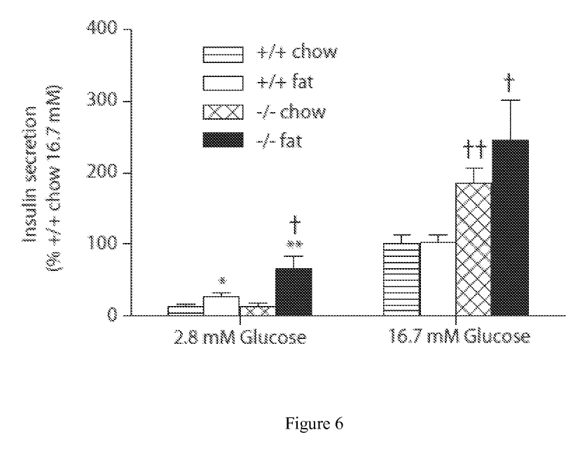

To investigate the role of Id1 in insulin secretion, GSIS was assessed in islets isolated from wild-type and Id1^{-/-} mice fed either a chow or a high-fat diet. Compared to chow-fed mice, insulin secretion at a low stimulatory level of glucose (2.8 mM) was significantly increased in islets isolated from fat-fed mice (FIG. 6). This fat diet-induced enhancement of insulin secretion at low glucose was greater in islets from Id1^{-/-} mice compared to wild-type controls. At a high stimulatory level of glucose (16.7 mM), insulin secretion in islets isolated from Id1^{-/-} mice was significantly increased compared to wild-type controls (FIG. 6), especially after high-fat feeding. Without being bound by any theory or mode of action, these results indicate that Id1 expression inhibits GSIS in mouse islets, particularly under conditions of lipid oversupply and/or insulin resistance. Again, without being bound by any theory or mode of action, the increased insulin levels in mice with Id1 inhibition appear to be due to enhanced insulin release from islets.

### Example 10

### Id1^{-/-} Mice are Protected Against Diet-Induced Loss of β-Cell Gene Expression

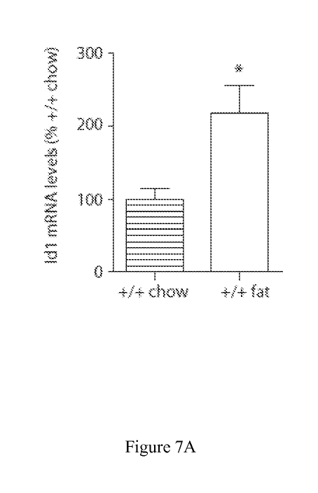

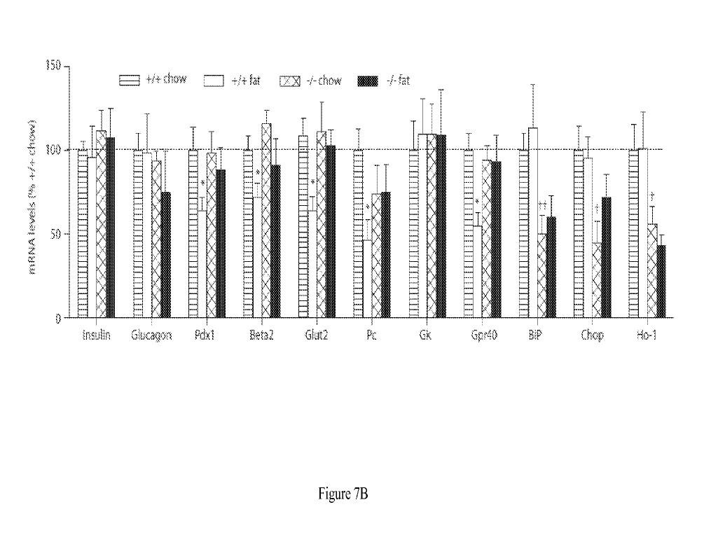

Expression of several genes involved in the maintenance and specialized function of the β-cell phenotype was also analyzed. mRNA levels were assessed in islets isolated from wild-type and Id1^{-/-} mice fed a chow or a high-fat diet. Id1 mRNA levels were increased by 2-fold in islets from fat fed mice compared to chow-fed controls (FIG. 7A). Id1 mRNA levels were undetectable in islets from Id1^{-/-} mice. Expression of the islet hormones, insulin and glucagon, were not affected by either diet or genotype (FIG. 7B). Pdx1 and Beta2 are transcription factors that are important for the maintenance of β-cell differentiation. mRNA levels of Pdx1 and Beta2 were significantly reduced in islets of fat-fed wild-type mice; Pdx1 was downregulated by ˜40% and Beta2 by ˜30% (FIG. 7B). However, in islets from fat-fed Id1^{-/-} mice, expression of Pdx1 and Beta2 were maintained at levels observed in chow-fed mice (FIG. 7B). The expression of several genes involved in β-cell glucose metabolism was also assessed. The glucose transporter, Glut2, and the anaplerotic enzyme, pyruvate carboxylase (Pc), were downregulated in islets of fat-fed wild-type mice, whereas these metabolic genes were not affected by high-fat feeding in Id1^{-/-} islets (FIG. 7B). Not all genes involved in glucose metabolism were altered: glucokinase mRNA levels were unchanged by diet or genotype. The G-protein-coupled receptor Gpr40 may play a role in both fatty acid and glucose stimulation of insulin secretion. Gpr40 expression was downregulated by ˜50% in fat-fed wild-type mice (FIG. 7B). In contrast, Gpr40 expression was unchanged after fat-feeding of Id1^{-/-} mice (FIG. 7B). These data demonstrate that islets from Id1^{-/-} mice are protected against high-fat diet-induced loss of β-cell gene expression.

### Example 11

### mRNA Levels of Stress Genes are Reduced in Islets of Id1^{-/-} Mice

Expression of genes associated with cellular stress and stress response mediators was also assessed. BiP is an endoplasmic reticulum (ER) chaperone and key regulator of the ER stress response and Chop is an ER stress-inducible transcription factor. Both BiP and Chop mRNA levels were significantly reduced in islets of Id1^{-/-} mice compared to wild-type controls (FIG. 7B). The antioxidant heme oxygenease-1 (Ho-1) is induced by oxidative stress. Ho-1 mRNA levels were significantly reduced in islets of Id1^{-/-} mice compared to wild-type controls (FIG. 7B). Moreover, mRNA levels for each of these stress genes were unchanged after high-fat feeding (FIG. 7B). These data indicate that reduced stress gene expression accompanies the augmentation of insulin secretion in islets from Id1^{-/-} mice.

### Example 12

### Overexpression of Id1 in MIN6 Cells Reduces Insulin Secretion

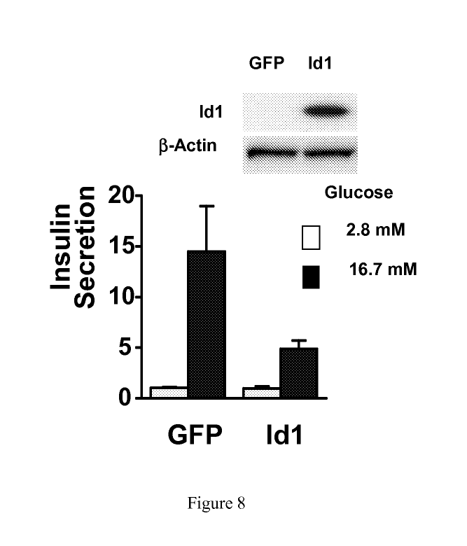

To examine the effects of increased Id1 expression on insulin secretion, murine Id1 cDNA was cloned into the expression vector pcDNA-DEST40 and transfected into MIN6 cells by nucleofector (Amaxa), and cells were assessed using an insulin secretion assay. While basal insulin secretion (2.8 mM glucose stimulation) was not altered, GSIS (16.7 mM glucose stimulation) was significantly reduced in Id1 overexpressing MIN6 β-cells compared to control GFP-expressing MIN6 β-cells (FIG. 8). This indicates that increased expression of Id1 in β-cells is sufficient to inhibit GSIS. This was not due to changes in insulin content or cell viability, which were not affected by Id1 overexpression in MIN6 cells.

### Example 13

### Id1 Plays a Role in Regulating Insulin Secretory Changes that Accompany Chronic Palmitate Exposure in MIN6 Cells

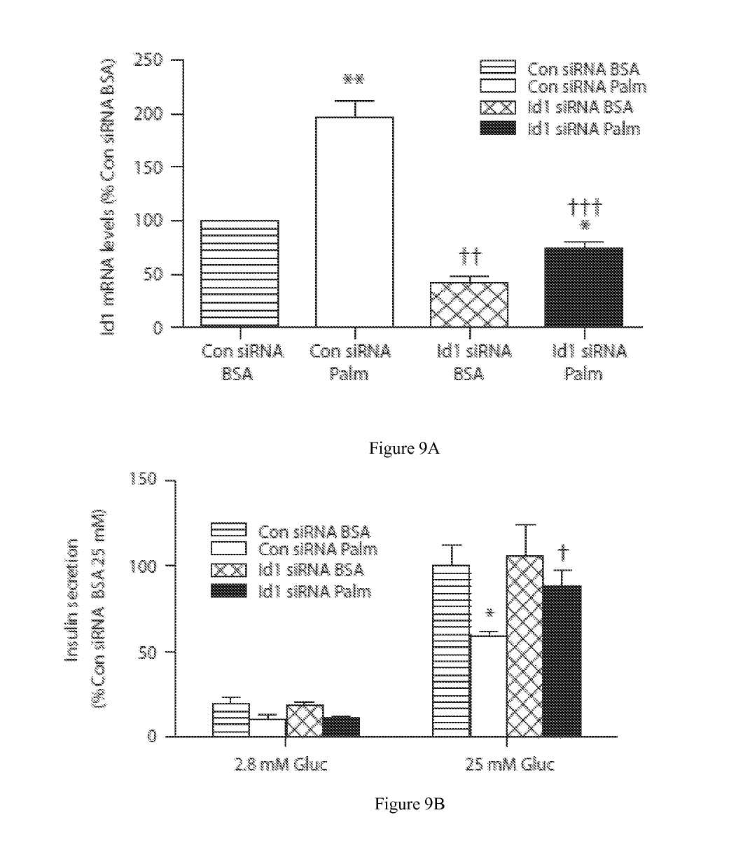

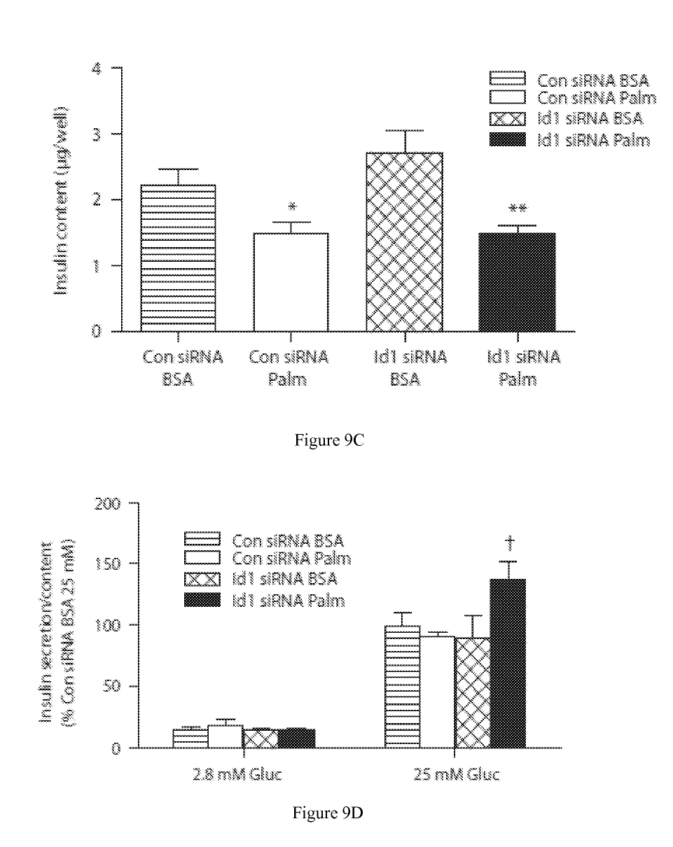

To examine the role of Id1 in insulin secretion in β-cells, the highly differentiated and glucose responsive mouse insulinoma β-cell line, MIN6 was used. Chronic exposure of MIN6 cells to elevated fatty acids has previously been shown to induce mild insulin secretory dysfunction and changes in gene expression consistent with a loss of β-cell differentiation. Exposure of MIN6 cells to the saturated fatty acid, palmitate (0.4 mM palmitate coupled to 0.92% BSA) for 48 h led to a 2-fold increase in Id1 mRNA levels (FIG. 9A). MIN6 cells were transfected with Id1 silencing siRNA or control siRNA. Id1 siRNA transfection led to reduced Id1 expression in MIN6 cells exposed to palmitate or BSA (FIG. 9A).

Insulin secretion was then assessed under these experimental conditions. In control siRNA-treated cells, chronic palmitate exposure significantly reduced the subsequent insulin secretory response to high glucose stimulation (FIG. 9B). In contrast, the insulin secretory response to high glucose stimulation was maintained in palmitate-treated MIN6 cells after knockdown of Id1 with siRNA (FIG. 9B). Cellular insulin content was also measured in these treatment groups. Cellular insulin content was significantly reduced in fatty acid treated cells (FIG. 9C). However, this was not affected by Id1 knockdown; chronic palmitate treatment led to a similar depletion of cellular insulin content in control siRNA- and Id1 siRNA transfected cells (FIG. 9C). The re-calculation of insulin secretion as a function of total insulin content negated the change in insulin secretion due to palmitate exposure in control siRNA transfected cells (FIG. 9D). These data emphasize the significantly increased insulin secretory response to high glucose stimulation in palmitate pre-treated MIN6 after knockdown of Id1 (FIG. 9D). Without being bound by any theory or mode of action, these data suggest that Id1 expression inhibits secretory function without affecting insulin depletion under conditions of increased lipid supply.

### Example 14

### Id1 Plays a Role in Regulating Gene Expression Changes that Accompany Chronic Palmitate Exposure in MIN6 Cells

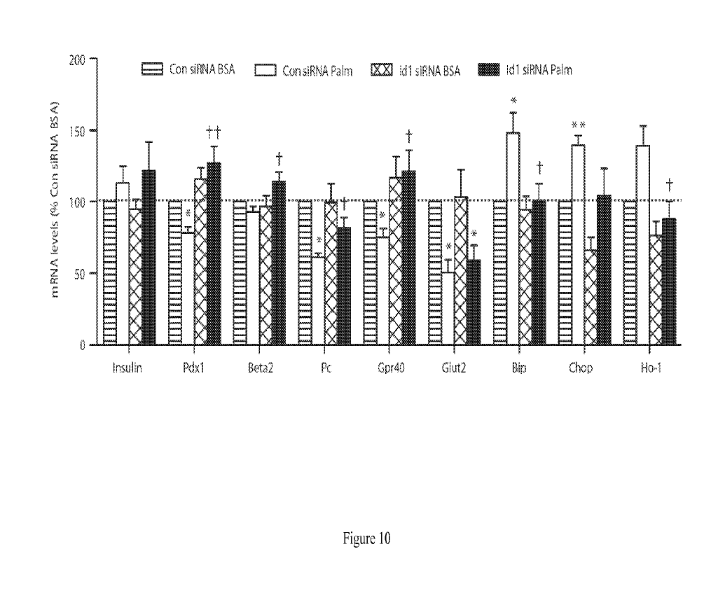

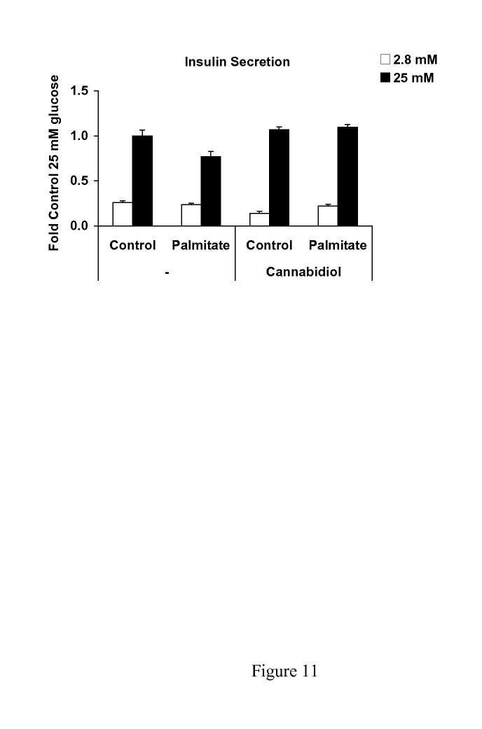

The role of Id1 in the regulation of gene expression in MIN6 cells was also assessed. Chronic (48 h) palmitate treatment of control MIN6 cells led to reduced expression of Pdx1, Pc, Gpr40 and Glut2, whereas insulin mRNA levels were not affected (FIG. 10). The knockdown of Id1 in MIN6 cells prevented the palmitate-mediated downregulation of Pdx1, Pc and Gpr40 (FIG. 10). However, Glut2 was not affected. Knockdown of Id1 in palmitate-treated cells significantly increased Pdx1 by ˜50%, Pc by ˜20% and Gpr40 by ˜40% compared to control palmitate-treated cells. Although not reduced by palmitate treatment in control MIN6 cells, Beta2 mRNA levels were significantly increased by ˜20% in palmitate-treated cells after knockdown of Id1 (FIG. 10). Without being bound by any theory or mode of action these results suggest that Id1 expression contributes to the downregulation of several important β-cell genes under conditions of chronic lipid oversupply.

### Example 15

### Treatment of Models of Type 2 Diabetes with Id1 Antagonists

### 15.1 Models of Type 2 Diabetes

One model of type 2 diabetes is produced by maintaining mice on a high fat diet, substantially as described above.

A second model is the high-fat diet-fed streptozotocin-treated (HFD-STZ) mouse, which is a non-genetic model of type 2 diabetes. Mice are fed a high-fat diet for 6 weeks followed by a moderate dose (ip injection 100 mg/kg) of STZ. This treatment combines insulin resistance and reduced β-cell mass, thus challenging the ability of remaining β-cells to survive and function.

A third model is the C57BL/KsJ db/db mouse model in which diabetes arises because of insulin secretory defects and β-cell apoptosis in the setting of time-dependent increases in obesity and insulin resistance.

### 15.2 Id1 Antagonists

One or more of the following antagonists is used.

1-(4-methoxybenzyl)-4-(2,4,5-trimethoxybenzyl)piperazine; N′-acetyl-N′-(3-chloro-4-methylphenyl)-2-hydroxy-2,2-diphenylacetohydrazide; 6,7-dimethoxy-2-methyl-1-oxo-3-pyridin-3-yl-N-[3-(trifluoromethyl)phenyl]-1,2,3,4-tetrahydroisoquinoline-4-carboxamide; 2-[3-(3,4-dimethylphenyl)-2,4-dioxo-3,4,6,7,8,9-hexahydro-2H-cyclohepta[4,5]thieno[2,3-d]pyrimidin-1(5H)-yl]-N-(2-methoxyethyl)acetamide; 4-acetyl-N,N-dibutyl-3,5-dimethyl-1H-pyrrole-2-carboxamide; N-(4-isopropylphenyl)-2-[8-morpholin-4-ylcarbonyl)-11-oxo dibenzo[b,f][1,4]thiazepin-10(11H)-yl]acetamide from Chemdiv Inc.

Ethyl 1-(3-[(2,5-dimethyloxyphenyl)amino]-3-oxopropyl)-4-piperidinecarboxylate hydrochloride; ethyl 2-amino-4-methyl-5-({[2-(trifluoromethyl)phenyl]amino}carbonyl)-3-thiophenecarboxylate; N-[3-)1,3-benzodioxol-5-yl)-3-(3-methoxyphenyl)propyl]-N-benzylpropanamide; methyl {7-[(3-methoxybenzyl)oxy]-4,8-dimethyl-2-oxo-2H-chromen-3-yl}acetate;

Maybridge Chemical Company (A division of Thermo Fisher Scientific) produces ethyl 3-hydroxy-5-methyl-6-oxo-1-phenyl-1,6-dihydropyrano[2,3-c]pyrazole-4-carboxylate 1-(1,3-benzodioxol-5-ylmethyl)-4-[(4-chlorobenzyl)sulfonyl]piperazine; N′-(4-isopropylphenyl)-1-benzothiophene-2-carbohydrazide from Chembridge Corporation.

5-(4-bromophenyl)-3-hydroxy-4-(3-methyl-4-propoxybenzoyl)-1-[2-(4-morpholinyl)ethyl]-1,5-dihydro-2H-pyrrol-2-one; 5-(4-fluorophenyl)-3-hydroxy-4-(4-isobutoxy-3-methyl)-1-[2-(4-morpholinyl]-1,5-dihydro-2H-pyrrol-2one; and 5-(3-bromophenyl)-4-(3-fluoro-4-methoxybenzoyl)-3-hydroxy-1-[3-(4-morpholinyl)propyl]-1,5-dihydro-2H-pyrrol-2-one from Sigma Aldrich.

Antisense comprising a sequence set forth in any one of SEQ ID NOs: 17-21, optionally conjugated to a pancreatic targeting peptide and/or a protein transduction domain using standard technologies.

A peptide antagonist of Id1 comprising a sequence set forth in any one of SEQ ID NOs: 5-16 or 28, optionally linked to a pancreatic targeting peptide and/or a protein transduction domain using standard technologies.

### 15.3 Treatment

Mice are administered one or more of the Id1 antagonists above during type 2 diabetes onset or after onset of detectable symptoms or after disease onset. Following treatment mice will be assessed for diabetes progression. Exemplary assays include blood collected by standard tail sampling—glucose measured by glucometer, insulin by ELISA), glucose tolerance (ipGTT), insulin secretion (ivGTT), insulin content (acid ethanol extraction), islet morphology (β-cell mass, insulin/glucagon immunostaining), proliferation (BrdU, Ki-67) and apoptosis (TUNEL, In Situ Cell Death Detection Kit, POD, Roche).

### Example 16

### An Inhibitor of Id1 Expression (Cannabidiol) Protects MIN6 Beta Cells Against Lipid (Palmitate)-Induced Insulin Secretory Dysfunction

The MIN6 cell line was also used to examine the effects of inhibition of Id1 on lipid-induced insulin secretory dysfunction. MIN6 cells were grown in DMEM. Cells were seeded at 2×10⁵ cells per well in 24-well plates. Cells were treated with either 0.92% BSA or 0.92% BSA coupled to 0.4 mM palmitate for 48 h, in combination with the absence or presence of 10 μM Cannabidiol. After chronic incubation, cells were washed in KRB buffer containing 2.8 mM glucose, and then they were preincubated for a further 30 min in 0.5 ml of the same medium at 37° C. This buffer was then replaced with 0.5 ml of prewarmed KRB containing 2.8 or 25 mM glucose, and it was incubated for a further 1 h at 37° C. An aliquot was then removed for analysis of insulin content by radioimmunoassay. As shown in FIG. 11, cannabidiol protected the cells against palmitate-induced insulin secretory dysfunction.

1. A method of treating or preventing an abnormality of glucose metabolism in a subject, the method comprising administering an antagonist of Inhibitor of Differentiation 1 (Id1) to the subject.

2. The method according to claim 1, wherein the subject suffers from a condition selected from the group consisting of type 2 diabetes, hyperglycaemia, hyperinsulinemia, insulin resistance, glucose intolerance and combinations thereof.

3. The method of claim 1, wherein the subject suffers from type 2 diabetes.

4. The method of claim 1, wherein the antagonist of Id1 is a small molecule, a nucleic acid, an antibody or a peptide.

5. The method of claim 1, wherein the antagonist binds to Id1.

6. The method of claim 5, wherein the antagonist is a peptide comprising a sequence set forth in any one or more of SEQ ID NOs: 5-16.

7. The method of claim 5, wherein the antagonist is a small molecule comprising a structure set forth in Formula I or a derivative or salt thereof: 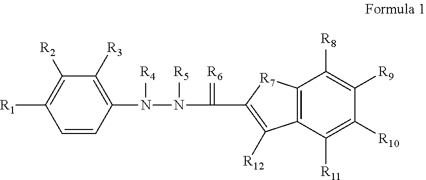
wherein R₁ is a substituted or unsubstituted lower hydrocarbon independently selected from the group consisting of alkyl, alkenyl, alkanoyl, alkynyl, aryl, aroyl, aralkyl, alkylamino, aryloxy, hydrogen, carboxyl, nitro, thioalkoxy, thioaryloxy, thiol, cycloalkenyl cycloalkyl, heterocycloalkyl, heteroaryl, aralkyl, amino acid, peptide, dye, fluorophore, carbohydrate or polypeptide; R₂ and R₃ are independently, collectively, or in any combination selected from hydrogen, hydroxyl, sulfyhydryl, fluorine, methyl, ethyl, propyl, benzyl, 2-bromovinyl amino, hydroxymethyl, methoxy, halogen, pseudohalogen, cyano, carboxyl, nitro, thioalkoxy, thioaryloxy, thiol, substituted or unsubstituted lower hydrocarbons containing 1 to 20 carbons, alkoxycarconyl, allkoxycarbonylamino, amino, amino acid, aminocarbonyl, aminocarbonyloxy, aralkyl, aryloxy, carboxyl, cycloalkenyl, alkyl, cycloalkyl, heterocycloalkyl, aryl, heteroaryl, aralkyl, amino acid, peptide, dye, fluorophore, carbohydrate or polypeptide; R₄ and R₅ are independently, collectively, or in any combination an acyl, or a substituted or unsubstituted lower hydrocarbon independently selected from the group consisting of alkyl, alkenyl, alkanoyl, aryl, aroyl, aralkyl or alkylamino; R₆ is oxygen, sulfur or nitrogen; R₇ is sulfur, nitrogen, oxygen or carbon; R₈, R₉, R₁₀, R₁₁ and Ri₂ are independently, collectively, or in any combination selected from the group consisting of hydrogen, hydroxyl, sulfyhydryl, fluorine, methyl, ethyl, propyl, benzyl, 2-bromovinyl amino, hydroxymethyl, methoxy, halogen, pseudohalogen, cyano and a substituted and unsubstituted lower hydrocarbon containing 1 to 20 carbons.

8. The method of claim 7, wherein the antagonist is a small molecule comprising a structure set forth in Formula II or a derivative or salt thereof:

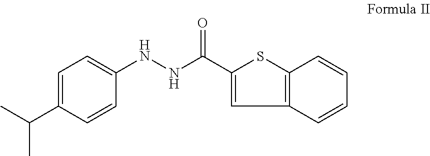 [SMILES: CC(C)c1ccc(NNC(=O)c2cc3ccccc3s2)cc1]

9. The method of claim 5, wherein the antagonist is a small molecule comprising a structure set forth in Formula III or a derivative or salt thereof: 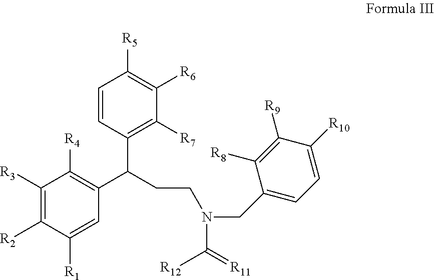
wherein, R₁, R₂, R₃, R₄, R₅, R₆, R₈, R₉, and R₁₀ may independently, collectively, or in any combination be selected from the group consisting of hydrogen, hydroxyl, sulfyhydryl, fluorine, methyl, ethyl, propyl, benzyl, 2-bromovinyl amino, hydroxymethyl, methoxy, halogen, pseudohalogen, cyano, carboxyl, nitro, thioalkoxy, thioaryloxy, thiol, substituted or unsubstituted lower hydrocarbon containing 1 to 20 carbons, alkoxycarconyl, allkoxycarbonylamino, amino, amino acid, aminocarbonyl, aminocarbonyloxy, aralkyl, aryloxy, carboxyl, cycloalkenyl, substituted or unsubstituted alkyl, substituted or unsubstituted cycloalkyl, substituted or unsubstituted heterocycloalkyl, substituted or unsubstituted aryl, substituted or unsubstituted heteroaryl, substituted or unsubstituted aralkyl, amino acid, peptide, dye, fluorophore, carbohydrate or polypeptide; R₇ is hydrogen, hydroxyl, benzoyl; substituted benzoyl or hydroxyl substituted with unsubstituted lower hydrocarbon containing 1 to 20 carbons; R₁₁ is oxygen, or another heteroatom such as sulfur or nitrogen; and R₁₂ is a substituted or unsubstituted lower hydrocarbon independently selected from the group consisting of alkyl, alkenyl, alkanoyl, alkynyl, aryl, aroyl, aralkyl, alkylamino, aryloxy, hydrogen, carboxyl, nitro, thioalkoxy, thioaryloxy, thiol, cycloalkenyl substituted or unsubstituted heteroatom, substituted or unsubstituted alkyl, substituted or unsubstituted cycloalkyl, substituted or unsubstituted heterocycloalkyl, substituted or unsubstituted aryl, substituted or unsubstituted heteroaryl, substituted or unsubstituted aralkyl, amino acid, peptide, dye, fluorophore, carbohydrate or polypeptide.

10. The method of claim 9, wherein the antagonist is a small molecule comprising a structure set forth in Formula IV or a derivative or salt thereof:

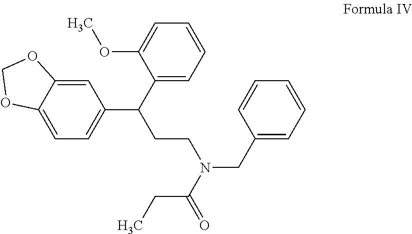 [SMILES: CCC(=O)N(CCC(c1ccc2c(c1)OCO2)c1ccccc1OC)Cc1ccccc1]

11. The method of claim 5, wherein the antagonist is a small molecule selected from the group consisting of: 1-(4-methoxybenzyl)-4-(2,4,5-trimethoxybenzyl)piperazine; N′-acetyl-N′-(3-chloro-4-methylphenyl)-2-hydroxy-2,2-diphenylacetohydrazide; 6,7-dimethoxy-2-methyl-1-oxo-3-pyridin-3-yl-N-[3-(trifluoromethyl)phenyl]-1,2,3,4-tetrahydroisoquinoline-4-carboxamide; 2-[3-(3,4-dimethylphenyl)-2,4-dioxo-3,4,6,7,8,9-hexahydro-2H-cyclohepta[4,5]thieno[2,3-d]pyrimidin-1(5H)-yl]-N-(2-methoxyethyl)acetamide; 4-acetyl-N,N-dibutyl-3,5-dimethyl-1H-pyrrole-2-carboxamide; N-(4-isopropylphenyl)-2-[8-morpholin-4-ylcarbonyl)-11-oxodibenzo[b,f][1,4]thiazepin-10(11H)-yl]acetamide; ethyl 1-(3-[(2,5-dimethyloxyphenyl)amino]-3-oxopropyl)-4-piperidinecarboxylate hydrochloride; ethyl 2-amino-4-methyl-5-({[2-(trifluoromethyl)phenyl]amino}carbonyl)-3-thiophenecarboxylate; N-[3-)1,3-benzodioxol-5-yl)-3-(3-methoxyphenyl)propyl]-N-benzylpropanamide; methyl {7-[(3-methoxybenzyl)oxy]-4,8-dimethyl-2-oxo-2H-chromen-3-yl}acetate; ethyl 3-hydroxy-5-methyl-6-oxo-1-phenyl-1,6-dihydropyrano[2,3-c]pyrazole-4-carboxylate; 1-(1,3-benzodioxol-5-ylmethyl)-4-[(4-chlorobenzyl)sulfonyl]piperazine; N′-(4-isopropylphenyl)-1-benzothiophene-2-carbohydrazide; 5-)4-bromophenyl)-3-hydroxy-4-(3-methyl-4-propoxybenzoyl)-1-[2-(4-morpholinyl)ethyl]-1,5-dihydro-2H-pyrrol-2-one; 5-(4-fluorophenyl)-3-hydroxy-4-(4-isobutoxy-3-methyl)-1-[2-(4-morpholinyl]-1,5-dihydro-2H-pyrrol-2one; and 5-(3-bromophenyl)-4-(3-fluoro-4-methoxybenzoyl)-3-hydroxy-1-[3-(4-morpholinyl)propyl]-1,5-dihydro-2H-pyrrol-2-one.

12. The method of claim 1, wherein the antagonist reduces or prevents Id1 expression.

13. The method of claim 12, wherein the antagonist is a nucleic acid that binds to Id1 encoding nucleic acid.

14. The method of claim 12, wherein the antagonist is a cannabidiol.

15. The method of claim 1, wherein the antagonist is linked to a compound that targets a pancreas in a subject.

16. The method of claim 1, wherein the antagonist is administered to the pancreas of the subject or to a blood vessel supplying a pancreas of a subject.

17. The method of claim 1, wherein the antagonist is administered in the form of a pharmaceutical composition.

18. A method of diagnosing or prognosing an abnormality of glucose metabolism in a subject, the method comprising detecting the level of expression of Inhibitor of Differentiation 1 (Id1), wherein an increased level of Id1 is diagnostic or prognostic of an abnormality of glucose metabolism in the subject.

19. The method of claim 18, comprising:
(i) detecting the level of expression of Inhibitor of Differentiation 1 (Id1); and
(ii) comparing the level of expression of Id1 to the level of expression of Id1 in a control sample,
wherein an increased level of Id1 is diagnostic or prognostic of an abnormality of glucose metabolism in the subject.
